(19) United States
(12) Patent Application Publication
(10) Pub. No.: US 2026/0325787 A1
(43) Pub. Date: Oct. 22, 2026

(54) AUTOMATED ACOUSTIC DELIVERY APPARATUS FOR WELLNESS APPLICATIONS AND METHODS THEREOF

(71) Applicant: Ajay SHARMA, Kolkata (IN)

(72) Inventor: Ajay SHARMA, Kolkata (IN)

(21) Appl. No.: 19/723,039

(22) Filed: Jun. 29, 2026

Related U.S. Application Data
(62) Division of application No. 19/452,234, filed on Jan. 17, 2026.

(30) Foreign Application Priority Data
Jan. 19, 2025 (IN) 202531004318

(51) Int. Cl. A61M 21/02; A61M 21/00

(52) U.S. Cl. CPC A61M 21/02; A61M 2021/0027; A61M 2205/10; A61M 2205/27; A61M 2205/3331; A61M 2205/502

(57) ABSTRACT

The present disclosure relates to automated acoustic delivery apparatus and method for wellness applications. The method comprises providing a plurality of acoustic instruments capable of producing distinct set of frequencies, arranging the plurality of acoustic instruments in a predetermined spatial configuration relative to a user, controlling at least one striking mechanism associated with the plurality of acoustic instruments. The controlling comprises defining an activation sequence specifying activation of each striking mechanism and a temporal order of activation, controlling strike force for activation of each striking mechanism, regulating temporal intervals between successive activations of each striking mechanism, and executing programmed sessions comprising multiple activations according to the activation sequence. The present disclosure addresses the limitations of manual sound therapy methods by reproducibly generating predetermined acoustic frequency relationships across the programmed sessions.

### FIELD OF THE DISCLOSURE

The present disclosure relates to the field of acoustic systems. More specifically, the present disclosure pertains to acoustic delivery apparatus and system for wellness applications.

### BACKGROUND OF THE DISCLOSURE

Sound therapy, often referred to as vibrational or acoustic medicine, has been used for centuries to promote physical, emotional, and spiritual well-being. Current meditation and relaxation devices typically rely on digital mediums, such as prerecorded audio files, synthesized soundscapes, or simple electronic tones. Digital reproduction often lacks the rich harmonic overtones and the tactile longitudinal pressure waves produced by physical instruments, which are frequently cited as critical components in resonant acoustic therapy and deep states of relaxation.

Acoustic delivery for wellness has been practiced for millennia across various cultures. Tibetan singing bowls, in particular, have been used for centuries in Buddhist meditation practices and are believed to promote relaxation, reduce stress, and facilitate well-being through their resonant acoustic properties. The bowls produce complex harmonic frequencies when struck or rubbed, creating vibrations that practitioners claim can realign energy, reduce tension, and induce deep relaxation states.

Contemporary research has begun to validate some traditional claims about acoustic wellness applications. Studies have demonstrated that specific acoustic frequencies can influence brainwave patterns through a phenomenon known as brainwave entrainment, wherein external rhythmic stimuli synchronize neural oscillations. For example, delta wave frequencies (0.5-4 Hz) are associated with deep sleep, theta waves (4-8 Hz) with meditation and creativity, alpha waves (8-13 Hz) with relaxed alertness, and beta waves (13-30 Hz) with active concentration. Acoustic stimulation at or near these frequencies, or that produces beat frequencies in these ranges, can potentially induce corresponding mental states.

Additionally, heart rate variability (HRV) has been shown to increase in response to relaxing acoustic stimulation, indicating enhanced parasympathetic nervous system activity and reduced stress response. Acoustic wellness applications have been explored for relaxation, stress management, meditation support, and general well-being, with evidence suggesting various physiological and psychological benefits.

Further, traditional sound healing and acoustic therapy rely almost exclusively on the manual manipulation of instruments such as singing bowls, gongs, or tuning forks by a practitioner. This manual approach is inherently subjective and prone to human error. Factors such as practitioner fatigue and inconsistent mallet velocity result in fluctuations in strike force and timing. This inconsistency prevents the delivery of a standardized therapeutic dose, making it difficult to maintain a steady environment conducive to meditation.

Manual methods cannot be standardized. There is no way to ensure that a “sleep support” session delivered by one practitioner on one day matches a session delivered by another practitioner or even by the same practitioner on a different day. Variations in strike force, timing, instrument selection, and spatial positioning are inevitable, making it impossible to establish evidence-based protocols.

Further, manual sessions cannot be precisely repeated. If a particular session produces beneficial effects, there is no reliable way to replicate the exact acoustic parameters in subsequent sessions. Practitioners cannot remember or reproduce precise timing intervals, strike forces, or frequency selections with the consistency required for systematic applications.

Manual acoustic delivery is inherently unscalable. Each session requires a trained practitioner's full attention for the entire duration, limiting the number of people who can receive sessions. This creates both access barriers (few practitioners relative to potential users) and cost barriers (practitioner time is expensive).

Wellness facilities such as spas, massage clinics, and meditation centers operate in leased spaces with limited budgets for structural modifications. Traditional acoustic therapy installations requiring custom-built furniture, permanent wall mountings, or structural alterations are impractical for most facilities. There exists a need for acoustic delivery systems that integrate into existing facilities without requiring expensive renovations or custom furniture, particularly systems that work with standard massage tables and treatment rooms already in place.

Existing systems that employ mechanical actuators to play musical instruments are predominantly designed for the fields of entertainment, automated composition, or robotic performance. These devices focus on the replication of complex melodies or the novelty of “player-less” instruments. However, these systems are not optimized for therapeutic outcomes, lacking the necessary sensory feedback loops or calibrated striking protocols required to induce specific physiological states or facilitate meditation.

A significant challenge in manual and basic mechanical sound production is the management of energy dissipation and harmonic interference. In manual practice, a practitioner cannot precisely control the rate of acoustic decay or the unintended interference patterns created when multiple instruments are struck in sequence. This often results in “muddy” or dissonant soundscapes where the therapeutic frequencies are masked by uncontrolled overtones or rapid energy loss, preventing the sustained resonance necessary for effective sound therapy.

Achieving complex spatial acoustic patterns or synchronized bilateral stimulation is a significant challenge in manual practice. Coordinating multiple instruments to produce specific phase relationships or moving sound fields requires a level of temporal precision that exceeds human capability. Furthermore, traditional mechanical actuators often introduce significant mechanical noise such as motor hum or clicking which creates auditory distractions that compromise the serene environment required for stress reduction.

Accordingly, there exists a profound need for an automated acoustic delivery system that can precisely regulate physical output while minimizing mechanical artifacts. Such a system must be capable of controlling the physics of sound production including strike force, interval timing, and spatial distribution, while managing harmonic interference and energy dissipation. By removing human variability and mechanical noise, an automated solution would allow for the standardization of acoustic therapy, enabling reproducible and data-driven wellness outcomes.

Further, current technological advancements in sound therapy are primarily limited to the automation of individual instruments. These solutions do not adequately address the need for programmable, customizable, and scalable sound therapy systems that integrate multiple instruments. The absence of such systems restricts the accessibility and affordability of sound therapy, limiting its widespread adoption.

The present disclosure addresses the limitations of manual sound therapy methods by providing automated acoustic delivery apparatus for wellness applications. The purpose of this disclosure is to deliver the benefits of sound therapy in a more accessible, consistent, and versatile manner.

### SUMMARY OF THE DISCLOSURE

In an embodiment of the present disclosure, an automated acoustic delivery apparatus for wellness applications is disclosed. The apparatus comprises a plurality of acoustic instruments having distinct set of frequencies of sound and at least one striking mechanism associated with at least one acoustic instrument. The at least one striking mechanism comprises a striking means configured to contact the associated acoustic instrument, an actuation mechanism configured to drive the striking means to strike the associated acoustic instrument with a programmable force, and a return mechanism configured to return the striking means from a striking position to a rest position after striking the associated acoustic instrument. The apparatus further comprises a processing unit operatively connected to each striking mechanism. The processing unit is configured to define an activation sequence specifying activation of each striking mechanism and a temporal order of activation, control strike force for activation of each striking mechanism, regulate temporal intervals between successive activations of each striking mechanism, and execute programmed sessions comprising multiple activations according to the activation sequence.

In an embodiment, the processing unit is configured to control strike force, timing, and spatial sequencing of the plurality of acoustic instruments to reproducibly generate predetermined acoustic frequency relationships across the programmed sessions.

In an embodiment, the apparatus further comprises a user interface communicatively coupled to the processing unit, the user interface configured to enable selection of session parameters of a programmed session. The session parameters comprise acoustic instrument selection, sequence of activation within the plurality of acoustic instruments, activation and pause timing in the sequence, and session duration

In an embodiment, the plurality of acoustic instruments are configured to deliver automated repeatable acoustic sessions with controlled frequency content, timing, and intensity for one or more applications comprising therapy, relaxation, wellness, meditation, stress reduction, and sound healing.

In an embodiment, the plurality of acoustic instruments comprises at least one Tibetan singing bowl, wherein each Tibetan singing bowl has a specifically engineered geometry and specific material compositions for generation of distinct set of frequencies of sound. The Tibetan singing bowls are interchangeable to accommodate distinct set of frequencies of sound. The processing unit is configured to activate the at least one Tibetan singing bowl to generate beat frequencies between the set of frequencies and harmonics thereof.

In an embodiment, each striking means comprises interchangeable striking heads comprising soft striking head configured to generate set of frequencies and lower-order harmonics when striking the associated acoustic instrument, and hard striking head configured to generate higher-order harmonics when striking the associated acoustic instrument.

In an embodiment, the plurality of acoustic instruments are arranged in a bilateral configuration or a multilateral configuration. The configuration comprises at least one acoustic instrument positioned on a left side relative to a user support surface at one or more levels, at least one acoustic instrument positioned on a right side relative to the user support surface at one or more levels, and/or at least one acoustic instrument positioned on at least one another side relative to the user support surface at one or more levels. The processing unit is configured to activate the left-side, right-side acoustic instruments, and the at least one another side acoustic instruments in a pattern selected from simultaneous bilateral or multilateral activation at the one or more levels, alternating bilateral or multilateral activation at the one or more levels, and sequential bilateral or multilateral activation at the one or more levels.

In an embodiment, the plurality of acoustic instruments are positioned in a spatial configuration comprising acoustic instruments at multiple distinct spatial locations relative to a user support surface, and wherein the processing unit is configured to execute spatial activation patterns utilizing the spatial configuration based on a type of application, wherein the type of application comprises therapy, relaxation, meditation, and stress reduction.

In an embodiment, the spatial configuration comprises one of acoustic instruments positioned on at least one side and on at least one level relative to the user support surface, and acoustic instruments positioned in a circular or elliptical arrangement in one or more levels surrounding the user support surface.

In an embodiment, the apparatus further comprises a mounting structure selected from one of: support tables and bed-mounted frames, wherein the plurality of acoustic instruments and the corresponding striking mechanisms are integrated with the mounting structure.

In an embodiment, the mounting structure comprises an adjustable positioning mechanism configured to position the plurality of acoustic instruments in a delivery configuration adjacent to a user during sessions, and retract the plurality of acoustic instruments to a storage configuration after session completion.

In an embodiment, the adjustable positioning mechanism comprises a hydraulic, pneumatic, or motorized adjustable positioning mechanism controlled by the processing unit.

In an embodiment, each striking mechanism further comprises a main striker bracket comprising a vertical link structure with a bearing housing providing rotational motion, that forms the striking means, a gravity shifting weight coupled to the main striker bracket serving as the return mechanism, and the actuation mechanism comprises an actuator selected from solenoid actuators, stepper motors, servo motors, and pneumatic cylinders, and an adjustable bar mounted on the actuator with a primary striker bearing configured to strike the main striker bracket.

In an embodiment, the vertical link structure rotates around a bearing of the bearing housing, a top part of the vertical link structure terminates in a bolted connection point, wherein the bolted connection point comprises the striking means, and a bottom part of the vertical link structure ends with a moulded silicon rubber or felt having a specific semi-spherical profile for receiving actuation force from the actuation mechanism.

In an embodiment, the primary striker bearing is configured to strike the moulded silicon rubber or felt (e.g. woolen, cotton, acrylic, polyester, etc.) at the bottom part of the vertical link structure to rotate the vertical link structure around the bearing, and to allow the striking means at the top part of the vertical link structure to strike the respective acoustic instrument, and the primary striker bearing is configured to detach from the moulded silicon rubber or felt before the striking means strikes the respective acoustic instrument.

In an embodiment, the apparatus further comprises at least one housing that houses the plurality of acoustic instruments along with the respective striking mechanisms. The housing comprises an openable cover board at a top for holding the plurality of acoustic instrument along with the respective striking mechanisms. The cover board comprises a plurality of layers/surfaces and the plurality of acoustic instruments are mounted on a top layer/surface of the cover board and the striking mechanisms are attached below a bottom layer/surface of the cover board.

In an embodiment, the cover board comprises at least one opening for at least one striking means, and wherein at least a portion of the striking means is present above the cover board and the remaining portion of the striking mechanisms is present below the cover board.

In an embodiment, each acoustic instrument is mounted on at least one hole present on the cover board using a fastening means, and wherein the at least one hole is selected for mounting the acoustic instrument, based on a size of the acoustic instrument to maintain a predetermined distance from the corresponding striking mechanism.

In an embodiment, the fastening means comprises a tension pin covered with a soft material, tension check nut for fastening the acoustic instrument at the bottom side of the cover board, and a string holder for holding the acoustic instrument at the top side of the cover board.

In an embodiment, the housing comprises sound dampening means on one or more interior surfaces and/or between the layers/surfaces of the cover board.

In an embodiment, the user interface comprises a mobile application executing on a wireless computing device, and wherein the mobile application is configured to communicate with the processing unit via wireless communication protocols.

In an embodiment, the apparatus is coupled with a cloud-based platform and the processing unit is configured to receive, from the cloud-based platform, force calibration data for each acoustic instrument and striking mechanism combination, the force calibration data relating actuator control parameters to resultant sound pressure level, and receive, from the cloud-based platform, force control logic for adjustment of actuator control parameters based on the force calibration data to achieve target sound pressure levels.

In an embodiment, the apparatus further comprises a memory. To execute programmed sessions, the processing unit is configured to retrieve library of session programs from the cloud-based platform for execution of the programmed sessions, and store the library of session programs in the memory for seamless operation.

In an embodiment, the apparatus further comprises a timer mechanism configured to automatically terminate sessions after a programmed duration, sound pressure level monitoring unit configured to maintain acoustic output below a predetermined maximum intensity threshold, and an emergency stop mechanism accessible to a user receiving sessions, configured to immediately halt all instrument activations upon activation.

In an embodiment, the apparatus further comprises a wireless communication interface configured to receive, from a remote computing device, commands for operating the apparatus, and transmit status information to the remote computing device.

In an embodiment, the apparatus further comprises a plurality of other acoustic instruments comprising one or more of: percussion instruments, chimes, bells, xylophones, flutes, and string instruments, and at least one actuation means for each of the other acoustic instruments, wherein the at least one actuation means configured to produce acoustic frequencies within a predefined range through the respective acoustic instrument.

In an embodiment, an automated acoustic delivery system for wellness applications is disclosed. The system comprises a plurality of automated acoustic delivery apparatus, a programmable controller operatively connected to plurality of automated acoustic delivery apparatus and configured to deliver standardized acoustic sessions across multiple apparatuses with consistent frequencies, timing, and intensity characteristics for one or more applications, a user interface communicatively coupled to the programmable controller, the user interface.

In an embodiment, the system is configured for retrofitting in at least one wellness facility. The system comprises a freestanding sub-surface unit configured to rest upon a floor surface and position beneath an existing user support structure without physically contacting the structure, and at least one peripheral unit positioned adjacent to the existing user support structure. The system integrates into at least one wellness facility without requiring structural modifications.

In an embodiment, a method for delivering automated acoustic stimulation for wellness applications is disclosed. The method comprises providing a plurality of acoustic instruments capable of producing distinct set of frequencies, arranging the plurality of acoustic instruments in a predetermined spatial configuration relative to a user, controlling at least one striking mechanism associated with the plurality of acoustic instruments. The controlling comprises defining an activation sequence specifying activation of each striking mechanism and a temporal order of activation, controlling strike force for activation of each striking mechanism, regulating temporal intervals between successive activations of each striking mechanism, and executing programmed sessions comprising multiple activations according to the activation sequence.

In an embodiment, the controlling comprises controlling strike force, timing, and spatial sequencing to reproducibly generate predetermined acoustic frequency relationships across the programmed sessions.

In an embodiment, the executing the programmed sessions comprises sending control signals to the striking mechanism to produce acoustic output according to the defined activation sequence, strike force parameters, temporal intervals, and session duration.

In an embodiment, the method further comprises delivering, using the plurality of acoustic instruments, automated repeatable acoustic sessions with controlled frequency content, timing, and intensity for one or more applications comprising therapy, relaxation, wellness, meditation, stress reduction, and sound healing.

In an embodiment, the method further comprises enabling selection of session parameters of a programmed session, and the session parameters comprise acoustic instrument selection, sequence of activation within the plurality of acoustic instruments, activation and pause timing in the sequence, and session duration.

In an embodiment, the plurality of acoustic instruments comprises at least one Tibetan singing bowl, each bowl producing a distinct set of frequencies, and the method further comprising selecting specific bowls based on their set of frequencies to achieve desired frequency characteristics for the wellness applications.

In an embodiment, the activation sequence comprises activating multiple acoustic instruments simultaneously to create complex harmonic patterns through interaction of the set of frequencies and harmonics from multiple sources for the wellness applications.

In an embodiment, the method further comprises arranging said instruments comprises positioning at least one acoustic instrument on a left side at one or more levels, at least one acoustic instrument on a right side relative to said user at one or more levels, and at least one acoustic instrument on at least one another side relative to the user at one or more levels; and activating the left-side, the right-side acoustic instruments, and the at least one another side acoustic instruments in a pattern selected from simultaneous bilateral or multilateral activation at the one or more levels, alternating bilateral or multilateral activation at the one or more levels, and sequential bilateral or multilateral activation at the one or more levels.

In an embodiment, the method further comprises selecting a left-side instrument with a set of frequencies having range F1, selecting a right-side instrument with a set of frequencies having range F2, wherein |F1-F2| falls within a target brainwave frequency range, and activating left-side and right-side instruments to generate bilateral beat frequency effects in the target brainwave frequency range for the wellness applications.

In an embodiment, the target brainwave frequency range comprises delta range, theta range, alpha range, and beta range.

In an embodiment, the method further comprises storing session parameters in a cloud-based platform, and enabling access to the stored session parameters by other systems for protocol replication across diverse wellness applications.

In an embodiment, the method further comprises controlling the plurality of acoustic instruments to gradually decrease activation frequency during the session, and transitioning from higher to lower brainwave frequency ranges.

In an embodiment, the method further comprises controlling strike force parameters to gradually increase from initial low intensity to target intensity during an initial portion of the session and gradually decrease from target intensity to low intensity during a final portion of said session for comfortable wellness applications.

In an embodiment, the arranging the plurality of acoustic instruments in the predetermined spatial configuration comprises positioning acoustic instruments on at least one side relative to the user, wherein the activation sequence comprises spatial patterns selected from: circular activation proceeding sequentially around said user, convergence activation proceeding from peripheral to central positions, and divergence activation proceeding from central to peripheral positions for the wellness applications.

In an embodiment, the method further comprises uploading session parameters associated with the programmed session to a cloud-based platform for sharing with other users for the wellness applications, and downloading protocol parameters from the cloud-based platform for storage and local execution.

In an embodiment, the method further comprises providing a candidate acoustic instrument, measuring a set of frequencies of the candidate acoustic instrument by: activating the candidate acoustic instrument with a standardized strike force, recording acoustic output with a calibrated microphone, performing frequency analysis to identify a set of frequency components, determining frequency stability by repeating said measuring for predetermined iterations and calculating a standard deviation of measured set of frequencies, measuring sound pressure level consistency by repeatedly activating said instrument with identical strike force and measuring sound pressure level variation, and selecting the candidate acoustic instrument for use in wellness applications if: frequency stability exhibits standard deviation of less than or equal to a frequency threshold, sound pressure level exhibits consistency within a predefined threshold value, and the candidate acoustic instrument exhibits no visible structural defects.

In an embodiment, the method further comprises documenting selection results in a specification sheet comprising measured fundamental frequency, frequency stability data, and sound pressure level characteristics, wherein the acoustic instruments are selected based on an objective performance criteria ensuring consistent output for wellness applications.

In an embodiment, the method further comprises measuring sustain duration by determining time elapsed from activation until acoustic output decays to a threshold level, and qualifying the candidate acoustic instrument only if sustain duration exceeds a predefined duration for use in wellness applications.

In an embodiment, the arranging the plurality of acoustic instruments in the predetermined spatial configuration comprises positioning at least one acoustic instrument beneath a user support surface, and wherein said activation delivers combined acoustic and vibrotactile stimulation through the user support surface to the user for wellness applications.

Other features and aspects of this disclosure will be apparent from the following description and the accompanying drawings. The features and advantages described in this summary and in the following detailed description are not all-inclusive, and particularly, many additional features and advantages will be apparent to one of ordinary skill in the relevant art, in view of the drawings, specification, and claims hereof. Moreover, it should be noted that the language used in the specification has been principally selected for readability and instructional purposes and may not have been selected to delineate or circumscribe the inventive subject matter, resort to the claims being necessary to determine such inventive subject matter.

### BRIEF DESCRIPTION OF THE DRAWINGS

The accompanying drawings, which are incorporated herein, and constitute a part of this disclosure, illustrate exemplary embodiments of the disclosed methods and systems in which like reference numerals refer to the same parts throughout the different drawings. Components in the drawings are not necessarily to scale, emphasis instead being placed upon clearly illustrating the principles of the present disclosure. Some drawings may indicate the components using block diagrams and may not represent the internal circuitry of each component. It will be appreciated by those skilled in the art that disclosure of such drawings includes disclosure of electrical components, electronic components or circuitry commonly used to implement such components.

FIG. 1 illustrates an environment for delivering automated acoustic stimulation for wellness applications, in accordance with some embodiments of the present disclosure.

FIG. 2 provides an internal block diagram of automated acoustic delivery system for wellness applications, in accordance with some embodiments of the present disclosure.

FIG. 3 shows the user interface screen layouts for session management, in accordance with some embodiments of the present disclosure.

FIGS. 4a & 4b depict the individual box design and setup of automated acoustic delivery apparatus, in accordance with some embodiments of the present disclosure.

FIG. 5 illustrates a detailed view of individual bowl striking mechanism, in accordance with some embodiments of the present disclosure.

FIG. 6a illustrates a detailed cross-sectional view of individual bowl striking mechanism, in accordance with some embodiments of the present disclosure

FIG. 6b-6f illustrate various interchangeable striking mallets and heads, in accordance with some embodiments of the present disclosure.

FIG. 7 shows driving components for operating an automated acoustic delivery apparatus, in accordance with some embodiments of the present disclosure.

FIG. 8a-8c illustrate bilateral and multilateral setup configurations around a user support surface, in accordance with some embodiments of the present disclosure.

FIG. 9 illustrates multi-level spatial arrangement of automated acoustic delivery apparatus, in accordance with some embodiments of the present disclosure.

FIG. 10 illustrates a block diagram of an automated acoustic delivery apparatus for delivering automated acoustic stimulation for wellness applications, in accordance with some embodiments of the present disclosure.

FIG. 11 illustrates a flowchart of a method for delivering automated acoustic stimulation for wellness applications, in accordance with some embodiments of the present disclosure.

The drawings are not necessarily to scale and may be illustrated by phantom lines, diagrammatic representations and fragmentary views. In certain instances, details that are not necessary for an understanding of the embodiments or that render other details difficult to perceive may have been omitted. The foregoing shall be more apparent from the following detailed description of the disclosure.

### DETAILED DESCRIPTION OF THE DISCLOSURE

Some embodiments of the present disclosure, illustrating all its features, will now be discussed in detail. It must also be noted that as used herein and in the appended claims, the singular forms “a”, “an” and “the” include plural references unless the context clearly dictates otherwise.

Various modifications to the embodiment will be readily apparent to those skilled in the art and the generic principles herein may be applied to other embodiments. However, one of ordinary skill in the art will readily recognize that the present disclosure including the definitions listed here below are not intended to be limited to the embodiments illustrated but is to be accorded the widest scope consistent with the principles and features described herein.

A person of ordinary skill in the art will readily ascertain that the illustrated steps detailed in the figures and here below are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments.

There exists a need for an acoustic delivery apparatus that can provide consistent and repeatable acoustic sessions without the variability inherent in human performance.

The terminologies ‘processing unit’, ‘controller’, ‘central controller’, and ‘programmable controller’ have the same meaning and are used alternatively throughout the specification.

The terminologies ‘user support surface’, ‘bed’, and ‘massage table’ have the same meaning and are used alternatively throughout the specification.

The following terms are defined to clarify and explain the scope and technical aspects of the present disclosure: Instruments refer to acoustic devices such as Tibetan singing bowls, percussion instruments, chimes, bells, xylophones, flutes, string instruments, and speakers, configured for individual or combined activation to produce therapeutic sound frequencies. Electro-mechanically controlled mallets are programmable devices associated with each instrument, capable of delivering varying force and duration by striking, with interchangeable heads. A programmable controller governs the sequence, duration, intensity, and activation patterns of instruments and mallets, enabling precise and repeatable sound sessions. The user interface, accessible via a software application, allows users to select pre-programmed sessions or design custom programs while connecting to a cloud platform for sharing, accessing, and monetizing sound sequences globally. Therapeutic sound frequencies, including Alpha, Beta, Theta, and Delta waves, are generated to promote relaxation and well-being. An optional bed-mounted device with hydraulics positions sound instruments alongside a bed during therapy sessions and retract them post-session, featuring a timer for automated management. Customizable parameters enable user-defined settings for sound sequences, mallet interactions, and instrument combinations, ensuring individualized and versatile therapeutic experiences. These definitions establish a comprehensive understanding of the disclosure's features and operational framework.

The present disclosure comprises an automated acoustic system that can be customized with various acoustic instruments, including Tibetan singing bowls of different sizes, percussion instruments, chimes, speakers, and like. The acoustic instruments are arranged in a configuration that allows for individual and combined activation. Each acoustic instrument is associated with an actuator, such as an electronically controlled mallet or any other activation means, capable of striking or activating the instrument with varying force and duration. The actuators are connected to a programmable controller or a processing unit that allows users to define the sequence, duration, and intensity of instrument activation. This enables the creation of a wide range of sound frequencies and patterns, including Alpha, Beta, Theta, Delta waves, or any combination thereof. The automated acoustic system can be controlled through a user interface, such as a mobile application, allowing users to select pre-programmed sound therapy sessions or create custom programs based on their needs. The application can be connected to a cloud-based platform.

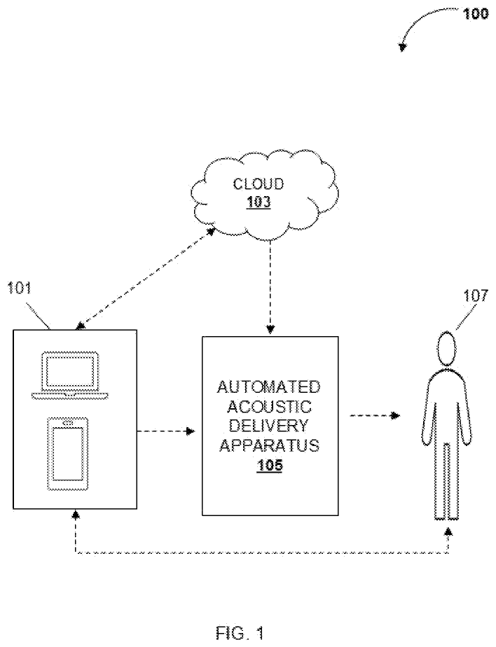

FIG. 1 illustrates an environment 100 for delivering automated acoustic stimulation for wellness applications, in accordance with some embodiments of the present disclosure.

In an embodiment of the present disclosure, the environment 100 may comprise a computing device 101, a cloud, 103, an automated acoustic delivery apparatus 105, and a user 107. The environment 100 may be a spa and wellness environment that is a carefully designed for fostering physical rejuvenation and mental tranquillity of the user 107.

The environment 100 relies heavily on sound/acoustic therapy to enhance the state of calm. Instead of abrasive noises of the modern world, the ambient soundscape is typically filled with rhythmic sounds of multiple acoustic instruments. These carefully curated sound profiles are generated strategically by placing acoustic instruments, and are designed to guide brainwave patterns toward meditative states like alpha and theta waves.

In an embodiment of the present disclosure, the environment 100 may comprise the computing device 101, the cloud 103, and the automated acoustic delivery apparatus 105 may be communicatively coupled with each other. In various embodiments, the communication network may be a wired network, a wireless network, or a combination thereof. Examples of networks suitable for use with the disclosed embodiments include, but are not limited to, a Local Area Network (LAN), a Wide Area Network (WAN) (e.g., the Internet), a cellular communication network (e.g., 4G, 5G, 6G), or other radio communication systems. In an embodiment, the computing device 101 and the automated acoustic delivery apparatus 105 may be connected through any other wireless communication protocol such as Bluetooth, Wi-Fi, and like.

A computing device 101 may be a wired or wireless device and may include, but is not limited to, a personal computer (PC), a laptop computer, a tablet computer, a smartphone, a mobile phone, a personal digital assistant (PDA), a media player, an e-book reader, a smart watch, a wearable computing device, a gaming console or platform, a server computer, a mainframe computer, a virtual machine, a distributed computing system, a cloud computing platform, an embedded system, a microcontroller, a system-on-chip (SoC), or any other device, apparatus, or arrangement of logic circuits capable of executing computer code or instructions. The specific hardware and/or software capabilities, input/output (I/O) devices (e.g., keyboard, mouse, display, touch screen, stylus, speakers, cameras), and storage media (e.g., solid-state drives, hard disk drives, RAM, ROM, memory chips, optical media, magnetic media) may vary depending on the specific implementation and desired functionality.

In an embodiment, the cloud 103 enables ubiquitous, convenient, on-demand network access to a shared pool of configurable computing resources (e.g., networks, servers, storage, applications, and services) that can be rapidly provisioned and released with minimal management effort or service provider interaction. In various embodiments, the present disclosure is implemented within a cloud computing environment that may include one or more private clouds, public clouds, or hybrid clouds. The cloud environment may utilize various service models, such as Software as a Service (SaaS), Platform as a Service (PaaS), and Infrastructure as a Service (IaaS), to provide the necessary functionality for the disclosed system and method.

In an embodiment, the cloud 103 may store session program libraries with activation sequences, manages user profiles and preferences, enables remote diagnostics and OTA updates, aggregates session data for analytics and feedback, and coordinates multi-device deployments across wellness facilities. However, the functionalities of cloud 103 is not limited to above example and any other facilities provided by cloud computing platform is well within the scope of present disclosure.

In an embodiment of the present disclosure, the automated acoustic delivery apparatus 105 overcomes the limitations of manual approaches. The automated acoustic delivery apparatus 105 delivers standardized, repeatable acoustic sessions without requiring manual practitioner intervention during session delivery, applicable to therapy, relaxation, wellness, meditation, stress reduction, sound healing, and other acoustic delivery applications.

In an embodiment, the automated acoustic delivery apparatus 105 may comprise a plurality of acoustic instruments arranged for individual or combined activation. The instruments include, but are not limited to, Tibetan singing bowls of various sizes and frequencies, percussion instruments, chimes, bells, xylophones, flutes, string instruments, and acoustic resonators. Each instrument is configured to produce distinct acoustic frequencies and harmonic content suitable for wellness applications.

The automated acoustic delivery apparatus 105 may regulate acoustic intensity, regulates temporal intervals between activation of different instruments with millisecond precision, and manages overall session duration with automatic start and stop functions.

The computing device 101 may comprise user interface, which may comprise a mobile application executing on a smartphone or tablet, or a dedicated hardware interface, enables users to select pre-programmed sessions from a library, design custom programs by specifying instrument combinations and activation parameters, and adjust session parameters including duration, intensity, and timing patterns. The interface communicates with the central controller of the automated acoustic delivery apparatus 105 via wired or wireless connection.

The automated acoustic delivery apparatus 105 governs the activation sequence, intensity, and duration for each instrument. The automated acoustic delivery apparatus 105 enables the creation of specific sound frequencies and patterns, such as Alpha, Beta, Theta, and Delta waves, which are crucial for achieving therapeutic effects. The automated acoustic delivery apparatus 105 may also facilitate the selection of pre-programmed therapy sessions or the customization of user-defined sequences and ensures seamless coordination of instrument activation to produce synchronized and harmonious soundscapes.

The automated acoustic delivery apparatus 105 integrates a user interface, also accessible via a mobile application, providing users with an intuitive platform to interact with the automated acoustic delivery apparatus 105. This interface allows users to choose from pre-programmed sessions or create and program custom sound sequences. It also supports real-time control and adjustment of actuators and instrument interactions. The integration of a cloud platform 103 further enhances the system's functionality by enabling users to share personalized sound therapy sessions with other users or therapists, access a global repository of pre-designed sound programs, and synchronize sessions for portability and cross-device compatibility, ensuring the automated acoustic delivery apparatus 105 is accessible from any location.

The automated acoustic delivery apparatus 105 addresses the limitations of manual sound therapy by offering an automated and programmable solution. Its ability to deliver consistent, scalable, and customizable therapy sessions makes it suitable for a wide range of applications, including clinical, personal, and professional environments.

Such an apparatus enables the implementation of standardized protocols with documented parameters, thereby ensuring reproducibility and reliability across different users and facilities. The system is designed to scale efficiently, serving multiple users without requiring proportional increases in skilled practitioners, and facilitates rigorous research by allowing precise control of acoustic interventions. By executing complex acoustic patterns that extend beyond manual capability, the apparatus reduces operational costs through elimination of per-session practitioner time. Furthermore, it comprehensively documents session parameters for scientific validation and quality assurance purposes, while also improving accessibility for populations lacking access to trained practitioners. Importantly, the apparatus is configured to integrate seamlessly into existing wellness facilities without necessitating structural modifications or specialized furniture, thereby supporting widespread adoption and deployment.

The components, implementations, and configurations of the automated acoustic delivery apparatus 105 are discussed in further detail below embodiments.

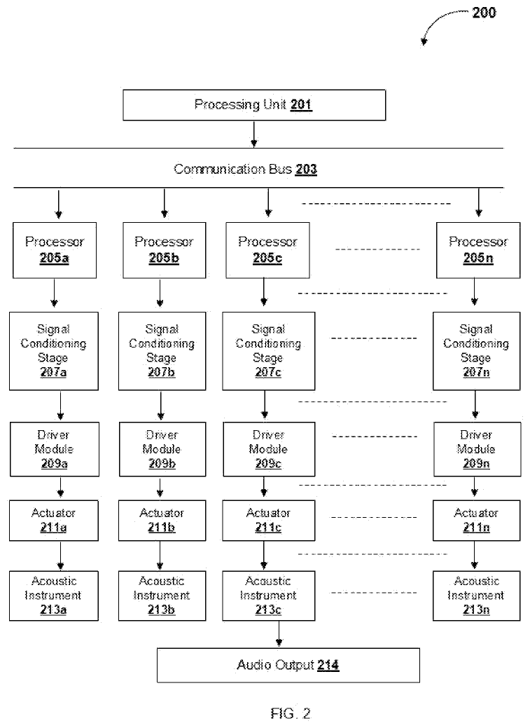

FIG. 2 provides an internal block diagram of automated acoustic delivery system 200 for wellness applications, in accordance with some embodiments of the present disclosure.

Referring to FIG. 2, a system 200 is disclosed for generating therapeutic audio output through coordinated digital synthesis and electro-mechanical excitation of acoustic instruments. The system comprises a primary processing unit 201, a communication bus 203, a plurality of processors 205a-205n, signal conditioning stages 207a-207n, driver modules 209a-209n, actuators 211a-211n, and acoustic instruments 213a-213n, culminating in a unified audio output 214. In one non-limiting embodiment, the system 200 may also comprise a graphical user interface (not shown) for control through the system and a mobile application (not shown) for remote selecting, scheduling, and executing programmed sessions.

In an embodiment, the processing unit 201 is configured to execute program session and transmit actuation commands for different acoustic instruments. The processing unit 201 may include one or more processors or microcontrollers, digital signal processing modules, scheduling logic for coordinating audio and mechanical events, and local/cloud storage for program definitions. Communication modules may support both wired and wireless protocols, and secure boot with encrypted transmission may be implemented. The program session executed by the processor may define actuator motion sequences, timing, tempo, amplitude, force parameters, and dynamic adaptation rules based on sensor feedback.

The communication bus 203 serves as the interface between the processing unit 201 and distributed actuator nodes. It may be implemented using differential serial protocols, Ethernet, or wireless channels. This bus facilitates synchronized distribution of control signals to multiple processors 205a-205n.

Each processor 205a-205n is associated with a distributed actuator node comprising several subsystems. The subsystems comprise a microcontroller unit executes firmware to decode commands from the processing unit 201, generates motion control pulses (e.g., step/direction, PWM, servo control), and monitors inputs such as limit switches, encoders, and force sensors.

The subsystems further comprise a signal conditioning stage 207a-207n: including transistor buffers, MOSFET drivers, optocouplers, digital isolators, logic-level translators, and EMI suppression components to ensure robust signal integrity. The subsystems comprise driver modules 209a-209n that drives various actuator types including stepper motors, servo motors, brushed/brushless DC motors, linear actuators, solenoids, and voice-coil actuators. These modules accept standard control inputs and deliver regulated current to actuators. However, the actuators for acoustic instruments are not limited to above examples and any other actuator known to a person skilled in the art, is well within the scope of present disclosure.

The subsystems comprise actuators 211a-211n that performs mechanical excitation such as striking, rubbing, plucking, tapping, bowing, or sustained vibration. These actuators interact with acoustic instruments 213a-213n including Tibetan bowls, drums, string instruments, chimes, bells, and resonant plates. However, the acoustic instruments are not limited to above examples and any other acoustic instrument for wellness application, is well within the scope of present disclosure.

The subsystems may also comprise mechanical interface may include mallets, plucking arms, rubbing pads, servo-driven plectra, and adjustable force/damping assemblies to facilitate precise mechanical interaction. The audio output 214 is synchronized with mechanical actuation to produce a cohesive therapeutic soundscape.

Thus, the system 200 provides standardized, repeatable acoustic sessions without requiring manual practitioner intervention during session delivery, applicable to therapy, relaxation, wellness, meditation, stress reduction, sound healing, and other acoustic delivery applications.

Further, the graphical user interface (GUI) may be provided via a touchscreen display. The GUI enables program selection, customization, session scheduling, real-time visualization, device diagnostics, OTA updates, and user authentication. Visual feedback may reflect actuator motion or audio envelopes.

In embodiment, GUI may enable selection of session parameters of a programmed session, wherein the session parameters comprise acoustic instrument selection of the plurality of automated acoustic delivery apparatus, sequence of activation within the plurality of acoustic instruments of the plurality of automated acoustic delivery apparatus, activation and pause timing in the sequence, and session duration.

In one non-limiting aspect, the user interface may be accessible to a user for only selecting a particular type of session e.g., session 1 for relaxation, session 2 for meditation . . . etc. In one non-limiting aspect, the user interface may be used by therapist or a professional specialized in the field of acoustics for modifying the session parameters. In one non-limiting aspect, the therapist or the professional may receive access for modification of session parameters from a network administrator.

Additionally, a mobile application may be used for remote control, including device pairing, program selection, personal and facility-based session scheduling, payment integration, session monitoring, and cloud-synchronized user preferences.

Furthermore, the cloud services may support centralized program libraries, multi-device facility management, analytics, telemetry, user profile storage, and remote diagnostics or updates.

In operation, the processing unit 201 loads a program session defining session parameters including acoustic instrument selection, sequence of activation within the plurality of acoustic instruments, activation and pause timing in the sequence, and session duration. Audio synthesis begins, and timed actuation commands are transmitted via bus 203 to processors 205a-205n. These processors coordinate motion profiles through driver modules 209a-209n and actuators 211a-211n, which interact with acoustic instruments 213a-213n resulting in physical sound sequence.

In one non-limiting embodiment, the audio output 214 may be analyzed and a feedback signal may be provided to the processing unit for standardized and repeatable acoustic session delivery.

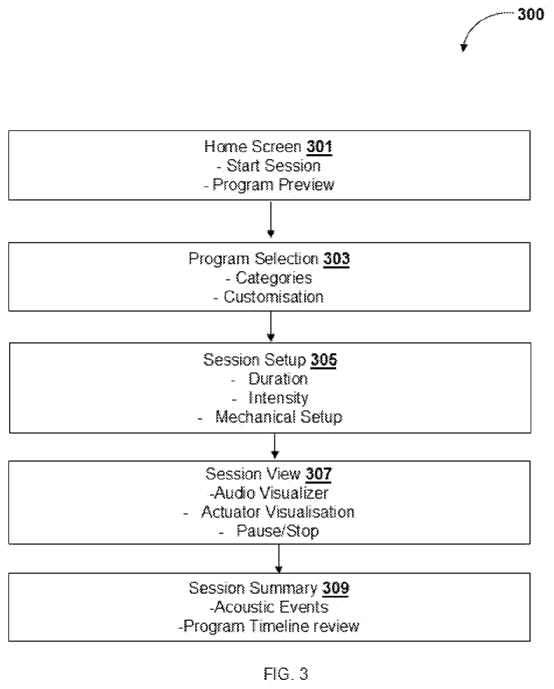

FIG. 3 shows exemplary user interface screen layouts for session management, in accordance with some embodiments of the present disclosure.

The block diagram of FIG. 3 depicts a sequential progression of screens and logic states that govern program initiation, configuration, execution, and post-session review. The system integrates audio generation, actuator control, and timeline management to deliver structured sessions tailored to user-selected categories, durations, intensities, and mechanical setups. Each stage—Home Screen 301, Program Selection 303, Session Setup 305, Session View 307, and Session Summary 309—implements discrete functions and data exchanges that ensure continuity, safety, and repeatability across sessions

The Home Screen 301 serves as the initial state of the application, presenting two principal actions: Start Session and Program Preview. Selecting Start Session initiates a guided workflow that advances to Program Selection 303, while Program Preview provides a non-committal overview of available categories, representative audio motifs, and actuator profiles without persisting configuration changes. In one embodiment, the Home Screen 301 also displays recent sessions, device connectivity status, and a quick-access control to resume a paused session, if supported by the underlying hardware and session state management.

Upon loading Home Screen 301, the system performs device discovery and readiness checks for audio output, apparatus, and actuators, etc. These checks may include verifying actuator firmware versions, that confirms safe operating ranges, and ensuring audio routing is available. If a device is unavailable or out of tolerance, the Home Screen 301 may present a notification and disable Start Session until the condition is resolved. In one example, a user connecting a new actuator module triggers an automatic calibration prompt before proceeding.

Transitioning to Program Selection 303, the user is presented with Categories and Customizations options. Categories define curated program families (e.g., relaxation, focus, recovery) with preconfigured audio envelopes and actuator sequences. Customizations allow the therapist or an acoustic expert to refine parameters within a selected category. The Program Selection 303 stage stores a provisional program profile that is passed forward to Session Setup 305. However, the above customizations options are exemplary and the system may provide other customizations as per user preferences.

The Session Setup 305 screen enables the user to define duration for the session. Duration may be specified in minutes or as a fixed number of cycles in the program timeline. The system computes derived parameters such as total acoustic events, and actuator cycles. In one embodiment, the system enforces minimum and maximum duration thresholds based on category presets and device safety constraints, presenting warnings if the user attempts to exceed recommended limits.

Within Session Setup 305, the intensity parameter governs audio amplitude, spectral content weighting, and actuator force or displacement profiles. Intensity may be expressed as a normalized scale (e.g., 1-10) or as discrete levels (e.g., low/medium/high). The system maps intensity to hardware-safe ranges and applies smoothing to avoid abrupt transitions during runtime. For example, selecting high intensity in a recovery program may cap actuator force at a conservative threshold while allowing richer audio harmonics to achieve perceived intensity without mechanical overdrive.

The Mechanical Setup portion of Session Setup 305 configures actuator selection, device/apparatus placement, orientation, and coupling to the target surface or accessory. The system may present recommended configurations based on the selected category, such as “edge mounting” for percussive programs or “central coupling” for resonant programs.

The Session View 307 is the operational screen where the session executes. It displays an audio visualizer that renders real-time amplitude, frequency bands, or waveform envelopes, and an actuator visualization that depicts actuator states, pulse timing, and cumulative cycles. The user can pause/stop the session at any time, triggering controlled ramp-down sequences to protect hardware and ensure a comfortable user experience. The system logs acoustic events and actuator actions with timestamps for later review.

In one non-limiting embodiment, the Session View 307 may allow limited mid-session edits, such as adjusting intensity within a safe range or toggling sub-features like “harmonic enrichment.” Edits are constrained to preserve mechanical setup assumptions and avoid destabilizing the session. The system records all adjustments, including timestamps and resulting parameter changes, to support accurate post-session analytics in Session Summary 309.

Upon completion or termination, the workflow advances to Session Summary 309, which presents acoustic events and program timeline review. Acoustic Events include counts and distributions of tones, sweeps, or pulses, along with peak and average levels. Program timeline review displays the executed sequence, highlighting segments, transitions, and any adaptive changes applied during runtime. The summary provides a concise yet comprehensive record of the session's behavior.

In one non-limiting embodiment, the Session Summary 309 may include compliance indicators, such as adherence to intensity limits and actuator duty cycles, and flags for any deviations or safety interventions. Users can export the summary as a report for documentation or share it with collaborators. In one embodiment, the system supports anonymized data export for research purposes, preserving program structure while omitting personal identifiers.

The user interface is modular, allowing new categories in Program Selection 303, additional calibration routines in Session Setup 305, and enhanced visualizations in Session View 307 without disrupting the core flow. In one example, a future update adds a “resonance mapping” feature that measures structural response during Mechanical Setup and automatically selects optimal actuator drive profiles. The modular design supports incremental improvements while maintaining backward compatibility.

The sequential design yields several technical effects: reduced configuration errors through staged validation, improved safety via preflight checks and adaptive control, enhanced user comprehension through real-time visualizations, and robust documentation via post-session analytics.

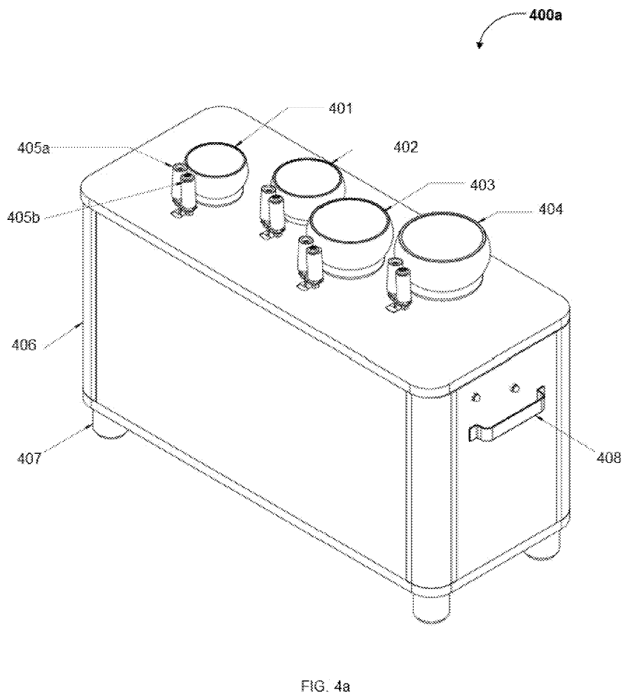

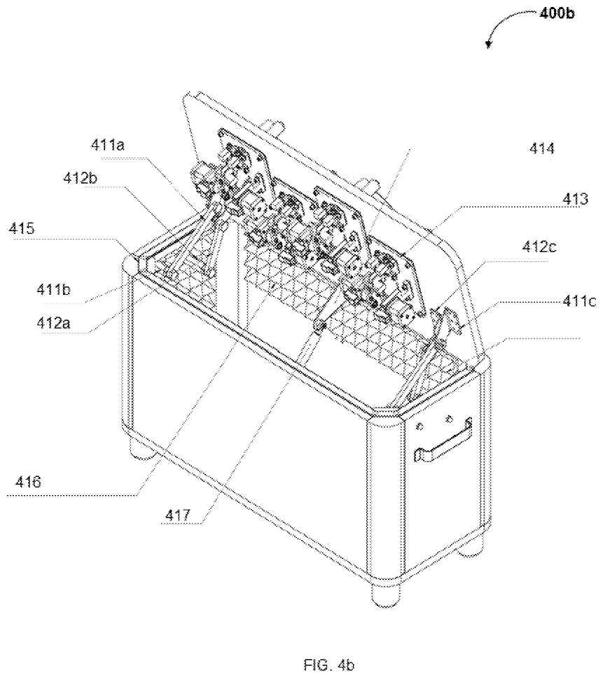

FIG. 4a depicts an external view of individual box design and setup of automated acoustic delivery apparatus 400a, in accordance with some embodiments of the present disclosure.

The automated acoustic delivery apparatus 400a comprises a rigid wooden box 406 capable of holding a plurality of acoustic instruments and their respective actuation devices. The external view of the box 406 illustrates 4 Tibetan singing bowls (or bowls) and 8 striking mallet configurations.

However, the present disclosure is not restricted to above mentioned configuration and may comprise any other configuration based on user preference, type of session, and environment of implementation of the automated acoustic delivery apparatus 400a.

The rigid wooden box 406 provides a stable structure and housing for Tibetan singing bowls and respective striking mechanisms, electrical, electronic as well as support for singing bowl.

In one non-limiting embodiment, the rigid wooden box 406 may be made of solid teak wood or marine plywood with high quality mica pasted on both inner and outer surface of the box 406. Further, to make the box user friendly, all the four corners are made with high quality teak wood having radius edges. This increases the stability and increased the life span of the box as well as it prevents accidentally hurting the user or anyone carrying it or passing by it. However, the present disclosure is not limited to above mentioned materials for rigid box 406 and any other materials having similar property may be used to implement the embodiments of the present disclosure.

In an embodiment, the box 406 may have at least four legs 407. In an example, the legs 407 may be made up of, but not limited to solid steel with powder coating. The legs 407 provide support to the rigid box and allow gap in floor surface and rigid box bottom surface. The legs 407 may elevate the rigid box bottom surface to a sufficient height to prevent exposure to moisture during cleaning of the environment and prevent deterioration of the wood materials used in the box. In one non-limiting embodiment, the legs may be replaced by wheel having appropriate locking mechanism for easy movement of box 406.

The box 406 may comprise at least one handle 408 for movement of the box 406. In an example, the handle 408 may be made up of, but not limited to, solid steel. The box 406 is having a modular design and it allows movement of the box 406 within a room or movement in different places. The lifting handles 408 are provided on two side of the box so that it can be lifted and moved at any place desired. The weight of the box is kept in such a way so that two people can comfortably lift the box and move anywhere needed.

The plurality of acoustic instruments comprises bowls 401, 402, 403, 404. Each bowl may have one or more striking mechanisms for generating a range of frequencies of sound. For example, the striking mechanisms 405a, 405b may be mapped to the bowl 401.

In an embodiment, the bowls 401-404 may be made up of bronze metal, bell metal, a metal composition including copper, tin, zinc, iron, lead, silver and gold. However, the material used in manufacturing bowl is not limited to above examples and any other type of singing bowl is well within the scope of present disclosure.

In an embodiment, the bowls 401-404 may be high quality bowls with specifically engineered geometry, specific material compositions and a well-defined process for accurate range of frequencies of bowl with same geometry. In an embodiment of the present disclosure, the bowls may have sound variability across a range of frequencies. The bowls may be activated in a manner to have repeatable acoustic sessions.

The bowls 401-404 are available in different sizes and different geometry having different resonating frequencies. The box 406 is designed in a manner that different size bowls can be mounted in all the 4 or more positions, as illustrated in FIG. 4a.

The bowls 401-404 are mounted on bowl support made with strong cotton cloth and inside it is filled with good quality cotton. In another, the bowl support cloth, coir, or a combination thereof. In center of the bowl support there is a small hole so that singing bowl holding string and pin can pass through the singing bowl, bowl support, plywood and mechanism main plate.

The design of the bowl support is flat in the center with at least 5 to 7 mm thickness and it is having medium hardness in the center. On the periphery bowl support is having doughnut shape which supports about periphery as well as it absorbs extra sound generated by the singing bowl. This bowl support design provides dual function of supporting the bowl from the center giving a smooth singing bowl experience. In one non-limiting embodiment, the bowl support may also absorb extra vibration generated by singing bowl.

In an embodiment, the striking mechanisms 405a, 405b may comprise mallets. The exterior surface of the mallet may be made up of, not limited to, teak wood, leather, soft furnishing fabric, soft foam, felt (e.g. woolen, cotton, acrylic, polyester, etc.). Thickness of the felt may determine the frequency pattern generated by the bowl. The internal surface of the mallet may be made up of, not limited to, teak wood, aluminum pin, solid Steel bush, rubber etc. For example, striking mallets are made of solid teak wood or any other long lasting hard woods, with leather or furnishing soft cloth as external surface. It can also have plain wood surface for more treble sound.

The function of the mallet is to strike on respective sound bowl and allow singing bowl to generate therapeutic sound. In an embodiment, the mallets replaceable for generating different harmonics sounds with different external and internal materials. Frequency of the bowl remains same but harmonics will change.

In an embodiment, each bowl may have at least one mallet. For example, one is hard and the other is soft. Each mallet is driven for strike with separate striking mechanism inside the box 406.

Mounted atop the main body 406 are four circular actuator interfaces, labeled 401, 402, 403, and 404, each corresponding to a distinct stimulation zone. These interfaces are symmetrically arranged to provide uniform spatial coverage and are constructed from acoustically transparent materials to facilitate sound propagation.

FIG. 4b illustrates an internal view of individual box design and setup of automated acoustic delivery apparatus 400b. The automated acoustic delivery apparatus 400b is similar to automated acoustic delivery apparatus 400a.

The internal view of individual box shows an openable cover board having a hinge link 411a, a hinge link 412a, a hinge pin 411b, a hinge pin 412b, a hinge bracket 411c, and a hinge bracket 412c along with gas spring hinge bracket 414, and gas spring 417 that allows smooth opening of the cover board. Further, the box comprises rubber or silicon lining 415 for sealing the box with cover board.

The gas spring 417 is selected in such a way that it keeps the top door assembly open at certain angle which is comfortable for assembling, dis assembling and maintenance of parts of top door assembly as well as panel components in bottom box. The design of hinges, top door assembly, hinge brackets, gas spring brackets combined function in such a way that it serves dual functions. One function is to keep the top door open when in open position and other is to keep the top door assembly slightly pressed against bottom box with rubber sealing surface to soundproof the box.

In an embodiment, the gas spring brackets 414, one on top door and another on bottom box inside wall. Both brackets have different design. Both the brackets have multiple holes to mount it on cover board and box surface and large base surface for adequate load transfer for keeping the box open in open condition without plywood deflection and keeping the top door slightly pressed on bottom box with rubber lining for making it soundproof.

In an embodiment, the hinge link 411a, the hinge link 412a may be made up of anodized aluminum, whereas the hinge pin 411b, the hinge pin 412b, the hinge bracket 411c, and the hinge bracket 412c may be made up of solid steel. They together provide top door hinge function in such a way that the box and the cover board can be sealed completely with rubber lining 415 for sound isolation of components present inside the box. In an example, for stable hinge function the hinge links 411a, 412b may provide special trajectory for the cover board opening. Along with gas spring, the cover board remains open for any maintenance or part replacement works and may be closed with downward force.

In an embodiment, the box comprises acoustic foam 416 inside box on all surfaces for extra sound absorption. The acoustic foam 416 may be of made up of high-density foam specially made for extra sound absorption. The acoustic foam 416 absorbs extra sounds generated by motors, moving parts in the assembly and provide noiseless experience to the user. The acoustic foam 416 leaves only audible sound of sound bowls. The acoustic foam 416 may cover majority inner surfaces for absorbing internal noises of actuation mechanisms.

In an embodiment the internal surface of the cover board further comprises automatic bowl striking mallet sub assembly 413. The number of the automatic bowl striking mallet sub assembly 414 may be variable and depends on number of bowls mounted on the box.

The automatic bowl striking mallet sub assembly 413 may be made up of multiple material assemblies such as aluminum, solid steel, wood, leather, cloth, silicon rubber, natural rubber etc. The automatic bowl striking mallet sub assembly 413 may provide modular design and endless flexibility of different configurations for different types of automated acoustic delivery apparatus and for different wellness applications. The modular design and identical sub-assemblies reduce the production cost, inventory, design variations, maintenance cost significantly.

In one non-limiting embodiment, the present disclosure is not limited to above discussed material types for different component and any other type of material known to a person skilled in the art that solves the purpose of present application is well within the scope of present disclosure.

FIG. 5 illustrates a detailed view of individual bowl striking mechanism 500, in accordance with some embodiments of the present disclosure.

FIG. 5 illustrates a front left isometric view 510a, a top view 510b, a front right isometric view 510c, a front view 510d, and right-side view 510e, in accordance with different embodiments of the present disclosure.

As shown, each bowl has two striking mallets and two mallet actuating mechanism with actuator with mounted primary striker. This modular design of individual bowl striking mechanism reduces the number of unique parts and sub-assemblies required for different models/configurations/variations of automated acoustic delivery apparatus. This ultimately reduces the cost of production, inventory and managing large number of part database, drawings, processes etc.

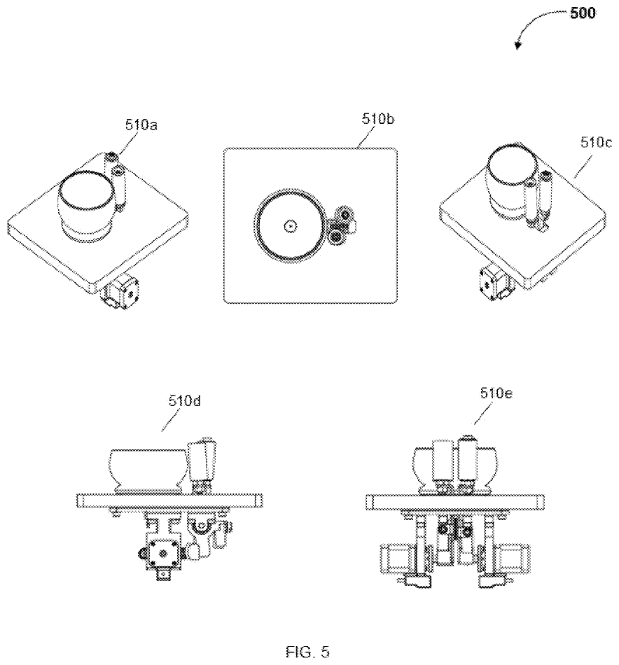

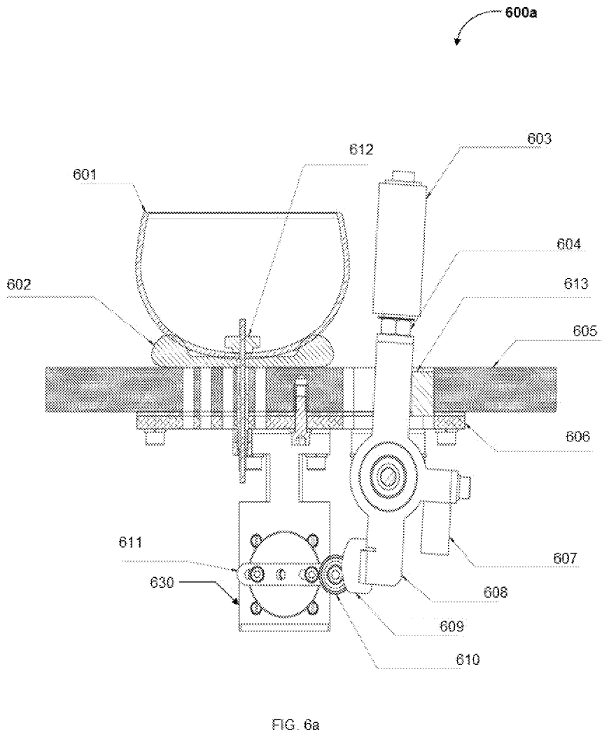

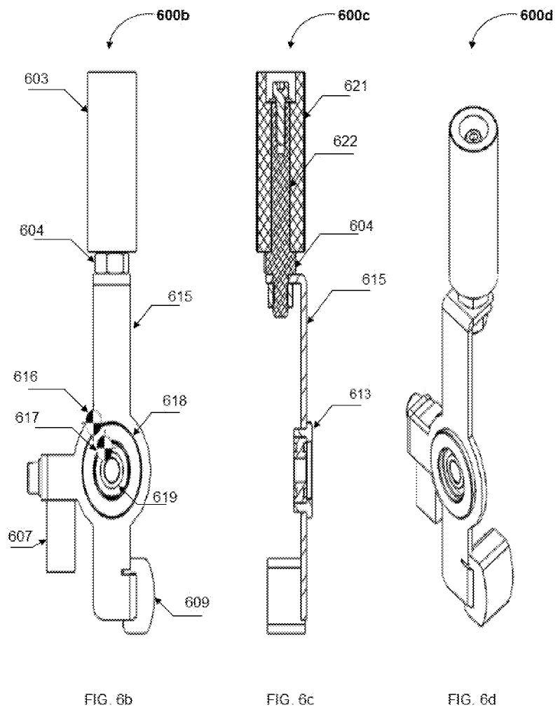

FIG. 6a illustrates a detailed cross-sectional view of individual bowl striking mechanism, in accordance with some embodiments of the present disclosure. In one non-limiting embodiment of the present disclosure, the below discussed mechanism can be extended to any number of individual bowl striking mechanism based on the programmed sessions and activation sequences described in the session parameters of the programmed sessions, as discussed in above embodiments. Further, the individual bowl striking mechanism 600a shows a single mallet operation and similar operation is applicable for one or more other mallets associated with the bowl.

The cross-sectional view of a mechanical assembly designated by 600a. This assembly 600a is configured to support and actuate a bowl 601 within a larger system, for acoustic application. The system comprises a vertically oriented actuation mechanism or striking mechanism integrated with structural supports and mechanical linkages.

The entire assembly 600a is mounted on the cover board 605 as discussed in explanation of FIGS. 4a and 4b. The assembly 600a is mounted on the cover board 605, using an aluminum plate 606 that extends below the cover board 605 and form a connection point of different sub-assemblies below the cover board 605. In one non-limiting embodiment, an anti-vibration rubber sheet between the cover board 605 and aluminum plate 606 is added to reduce the vibration and noise in the striking mechanism.

In an embodiment of the present disclosure, the cover board 605 comprises at least one opening for at least one striking mechanism. At least a portion of the striking mechanism is present above the cover board 605 and the remaining portion of the striking mechanisms is present below the cover board 605.

As shown in FIG. 6b, the striking mechanism comprises a main striker bracket 615 comprising a vertical link structure with a bearing housing 618 providing rotational motion. The main striker bracket 615 forms the striking means. The striking mechanism comprises a gravity shifting weight 607 coupled to the main striker bracket 615 serving as the return mechanism for the main striker bracket 615. The main striker bracket 615 may rest on resting pad 613 due to the gravity shifting weight 607. Further, the main striker bracket 615 is at a predetermined angle at a rest position from the vertical plane. The vertical link structure rotates around a bearing 619 of the bearing housing 618. The bearing housing 618 may be coupled to the bottom surface of the cover board 605 such that main striker bracket 615 is connected to the cover board using the aluminum plate 606 and anti-vibration rubber sheet. A top part of the vertical link structure terminates in a bolted connection point 604 and the bolted connection point 605 comprises the mallet 603. A bottom part of the vertical link structure ends with a moulded silicon rubber or felt (e.g. woolen, cotton, acrylic, polyester, etc.) 609 having a specific semi-cylindrical or circular profile for receiving actuation force from an actuation mechanism.

As shown in FIG. 6b, the bowl 601 and a bowl support is mounted on at least one hole present on the cover board using a fastening means 612. The at least one hole is selected for mounting the acoustic instrument, based on a size of the acoustic instrument to maintain a predetermined distance from the corresponding striking mechanism. The fastening means 612 comprises a tension pin covered with a soft material, tension check nut for fastening the acoustic instrument at the bottom side of the cover board, and a string holder for holding the acoustic instrument at the top side of the cover board 605.

The striking mechanism comprises an actuation mechanism that comprises an actuator selected from solenoid actuators, stepper motors, servo motors, and pneumatic cylinders, and an adjustable bar 611 mounted on the actuator with a primary striker bearing 610 configured to strike the main striker bracket 615.

In an embodiment, in response to an actuation from the actuator, the primary striker bearing 610 may strike the moulded silicon rubber or felt 609 at the bottom part of the vertical link structure to rotate the vertical link structure around the bearing 619, to move the main striker bracket from the rest position to a striking position and allow the mallet 603 at the top part of the vertical link structure to strike the bowl 601. The primary striker bearing 610 is configured to detach from the moulded silicon rubber or felt before the mallet 603 strikes the bowl, thereby generating set of frequencies of sound. In one non-limiting embodiment, the set of frequencies may comprise fundamental frequency and associated harmonic components within a measurable acoustic bandwidth,

As shown in FIG. 6c, the mallet 603 may comprise a mallet holding pin with rubber lining 622 that is bolted to the main striker bracket 615. The mallet 603 further comprises hardwood center 621 having an outer cover made up of cotton, wool, soft cloth, leather etc. In one non-limiting embodiment, the mallet 603 may be made up of plain hardwood covered with cotton material, leather, or felt (e.g. woolen, cotton, acrylic, polyester, etc.).

FIG. 6d illustrates a front isometric view of the striking means. In one non limiting embodiment, the mallet 603 may be replaced by wire loop beater 623, as shown in FIG. 6e. In one non limiting embodiment, the mallet 603 may be replaced by hand percussion instrument 625 for different acoustic applications, as shown in FIG. 6f.

In one non-limiting embodiment, the actuator sub-assembly 630 may be mounted using rubber padding to dampen the vibration generated by the actuator and to reduce the noise generated by the vibrations and micro vibrations of the actuator. This rubber padding isolates the actuator and primary striker bearing 610 having a rubber padding, and doesn't allow actuator vibrations to transfer to other parts of the assembly. This rubber padding is mounted between actuator and actuator mounting bracket.

In one non-limiting embodiment, the actuator sub-assembly 630 may comprise a motor can rotate at different RPM (Revolutions per minute) for fast or slow strike of the mallet 603 depending on programmed activation sequences. Various technical details like acceleration, deceleration, RPM of the motor can be controlled with the program. Based on different sound and vibration generation requirements, the motor will rotate at different speeds. This will make different sound and vibrations.

In an embodiment, the motor may be mounted using a mounting plate made of anodized aluminum that is designed in such a way that the individual motor assembly with primary striker bearing, adjustable bar, flange on motor shaft along with the motor mounting plate becomes a modular assembly. The motor mounting plate is mounted with anti-vibration rubber pads on both main plate and motor side.

In one non-limiting aspect, the striking mechanism may comprise a proximity sensor for homing and is used for stopping the actuator such as motor exactly at the starting angle position. The function of stopping the motor at the same starting angle is crucial for repeatable sound and vibration generated with sound bowl. The proximity sensor senses the edge of the adjustable bar 611 and stops the motor rotation exactly at the same angle position repeatably based on program from programmed card, PLC etc.

In an embodiment, the mallet 603 may be soft striking mallet and comprises a hardwood covered with soft cloth or felt (e.g., woolen, cotton, acrylic, polyester, etc. There is a hole in the center of wooden mallet. With this hole the wooden mallet fits on to the pin and the main striker bracket 615. The pin is grooved to fill rubber and the slight gap between wood and pin is also filled with rubber. This stabilizes the wooden mallet and dampens extra vibration generated in the assembly.

In an embodiment, the mallet 603 may be hard striking mallet and comprises a hardwood covered with leather/plain hardwood mallet. There is a hole in the center of wooden mallet. With this hole the wooden mallet fits on to the pin and striker bracket 615. The pin is grooved to fill rubber and the slight gap between wood and pin is also filled with rubber. This stabilizes the wooden mallet and dampens extra vibration generated in the assembly.

In one non-limiting embodiment, one or more silicon rubber washer fits may be used across various bolted connections within the individual bowl striking mechanism.

In one non-limiting embodiment, the resting pad 613 for main striker bracket 615 provide precise rest position of the moulded silicon rubber felt (e.g. woolen, cotton, acrylic, polyester, etc.) 609 with circular profile. Precise resting position ensures when the primary striker bearing 610 hits the silicon rubber or felt the striking assembly generates repeatable and uniform sounds.

In one non-limiting embodiment, the cover board 605 may comprise fitted heli-coils which fits inside the plywood of the cover board with thread and araldite. The locking screw for all mechanism of thread fits into the heli-coils. This way the heli-coils and plywood are firmly joined together. The screws used for fitting different mechanisms with plywood won't come in direct contact with the plywood. The screws will fit into the heli-coils. This will significantly increase the life of plywood and allow assembly and removal without any damage to the plywood.

In one non-limiting embodiment, the gravity shifting weight 607 is used to keep the main striker bracket 615 in exact resting position by gravity and bring back the main striker bracket 615 into resting position after mallet 603 striking the sound bowl 601. This weight may be precisely calibrated based on the weight of the main striker bracket 615 and weight of the mallets. This is one of the key components in generating repeatable sound and vibration from sound bowl. In one non-limiting embodiment, the gravity shifting weight 607 may shift the center of gravity from position 617 to 616 for resting the main striker bracket 615 at the resting pad 613.

In one non-limiting embodiment, the main striker bracket is made of Solid Steel 304 (SS 304). It is non corrosive material. There is a hole in the center of bracket for round bearing hosing 618 fitting and it is welded. The bearing 619 is tightly and precisely fit in the center. This bearing 619 allows angular rotation of the bracket in forward and reverse direction. The bracket 619 is moulded with specially shaped circular profile silicon rubber or felt (woolen, cotton, acrylic, polyester, etc.) on the bottom side. The silicon rubber or felt of 45 Shore A hardness is selected for sound dampening, extremely long life of the rubber, non-deterioration capabilities against all weather conditions and anti-abrasive and stable physical dimension holding capacity. The main striker bracket 615 may moulded with small silicon rubber or felt where it touches the resting pad 613 for keeping long life of both silicon resting pad and main striker bracket 615.

In one non-limiting embodiment, the primary striker bearing 610 may have a strike beginning angle with circular surface of the moulded silicon rubber or felt on main striker bracket 615. This angle should by design or be manually adjusted to angle of 15 degree or less. This is important for lower noise generation on strike.

In one non-limiting embodiment, the primary striker bearing 610 have a strike relieving angle with circular surface of the moulded silicon rubber or felt on main striking bracket. This angle should be 10 Degree or less by design. The primary striker bearing 610 should be relieved from circular surface of the moulded silicon rubber or felt before the mallet strikes sound bowl for effective sound and vibration generation. This is one of the most important design elements of the main striker bracket 615.

In an embodiment of the present disclosure, the adjustable bar 611 is drilled and tapped with M5 threaded hole for mounting of the primary striker bearing 610 with M5 screw. This bar is having 2 capsule slots for locking and adjusting distance between center of the flange on actuator shaft and center of the primary striking bearing. As we increase or decrease this distance the strike begins angle and strike relieve angle changes. The mallet strike velocity increases and decreases respectively with change in this distance.

In one non-limiting embodiment, the bowl tension check nuts are manually adjusted to increase or decrease the tension of silicon rubber string. The sound bowl 601 is drilled with a hole in the center to pass through this string and bowl tension pin. The sound bowl 601 slightly moves when the striking mallet strikes it. This movement is intentionally allowed with the flexible silicon rubber string. Due to tension in the silicon rubber string, the bowl 601 retains its original position after the mallet 603 strikes. This tension is critical for proper sound and vibration generation with sound bowl.

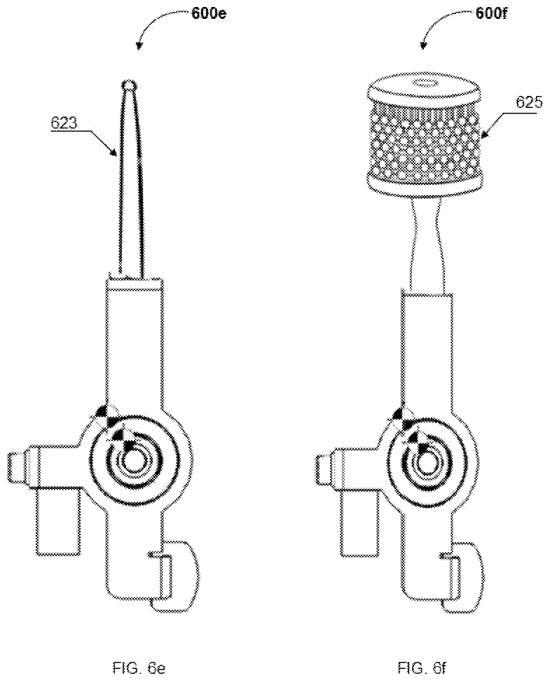

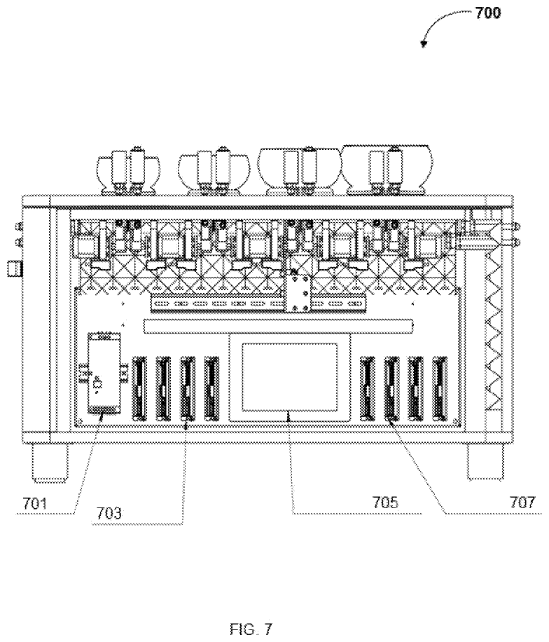

FIG. 7 shows driving components for operating an automated acoustic delivery apparatus 700, in accordance with some embodiments of the present disclosure.

In an embodiment of the present disclosure, the driving components may include a power unit 701, a first set of actuator drivers 703, controller 705, and a second set of actuator drivers 707. In one non-limiting embodiment, the power unit 701 may be configured to provide 24 V Switched-Mode Power Supply (SMPS) located at the lower section of the assembly. The SMPS converts AC input to regulated DC output, providing stable power to all electronic components of the automated acoustic delivery apparatus 700. The integration of the SMPS, actuator drivers 703, 707, and controller within a compact enclosure facilitates modular deployment.

In an embodiment of the present disclosure, the controller 705 or processing unit may comprise a centrally located Raspberry Pi-based programmed card serves as the primary control unit, executing preloaded instructions and managing signal distribution to the actuator drivers 703, 707. This microcontroller platform is configured to interface with external sensors, actuators, and user-defined input protocols, allowing for flexible automation and real-time feedback control.

In one non-limiting embodiment, the actuator drivers 703, 707 comprises stepper motor drivers, positioned on both lateral sides of the enclosure. These drivers regulate current and voltage supplied to each actuator such as “stepper motor”, ensuring smooth operation and protecting against overload conditions. The actuator drivers 703, 707 are connected via shielded wiring to the control card and motors, forming a closed-loop system capable of executing complex motion sequences with high repeatability.

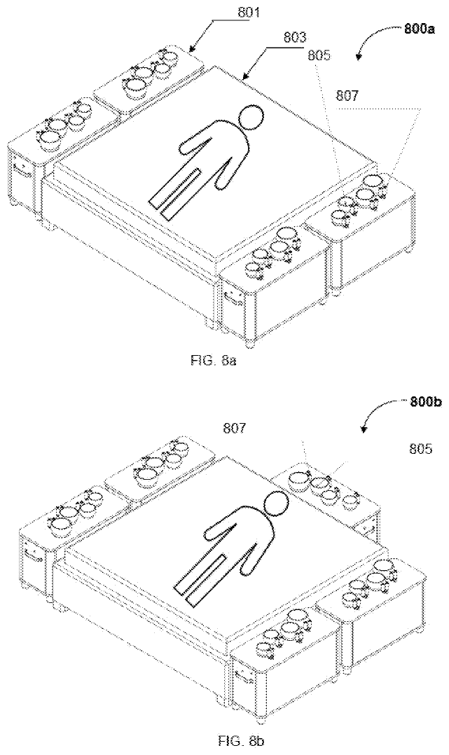

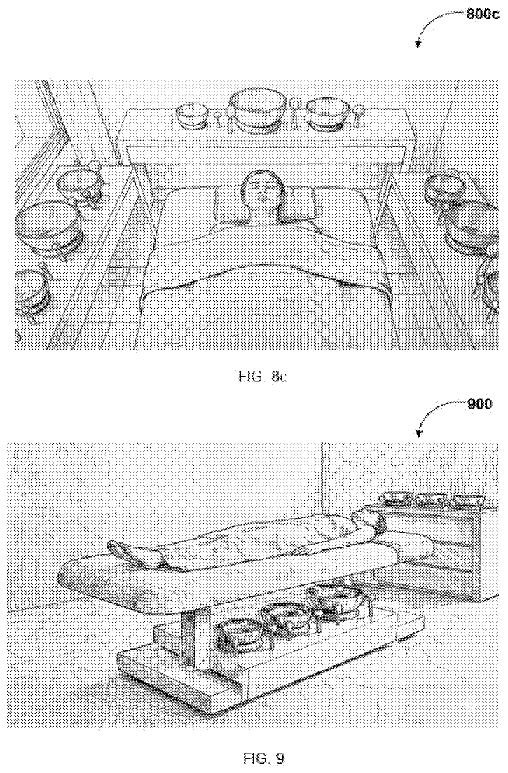

FIGS. 8a, 8b, 8c illustrate bilateral and multilateral setup configurations around a user support surface, in accordance with some embodiments of the present disclosure.

As shown in FIG. 8a, a user may lie on a user support surface 803 and a bilateral configuration of automated acoustic delivery apparatus 801 may be arranged relative to the i.e. on left side and right side of the user support surface 803. Each automated acoustic delivery apparatus may comprise at least one bowl 805 and associated one or more mallets 807. However, the present invention is not limited to above example and any other acoustic instruments may be part of the automated acoustic delivery apparatus.

As shown in FIGS. 8b and 8c, a user may lie on a user support surface 803 and a multilateral configuration of automated acoustic delivery apparatus 801 may be arranged relative to the i.e. on left side, right side, and any other remaining side of the user support surface 803. Each automated acoustic delivery apparatus may comprise acoustic instrument such as at least one bowl 805 and associated one or more mallets 807. However, the present invention is not limited to above example and any other acoustic instruments may be part of the automated acoustic delivery apparatus.

In an embodiment, a central processing unit may operate the automated acoustic delivery apparatus in a pattern selected from simultaneous bilateral or multilateral activation at the one or more levels, alternating bilateral or multilateral activation, and sequential bilateral or multilateral activation.

FIG. 9 illustrates multi-level spatial arrangement 900 of automated acoustic delivery apparatus, in accordance with some embodiments of the present disclosure.

In one non-limiting embodiment, the automated acoustic delivery apparatus may be arranged at multiple level along with unilateral, bilateral, or multilateral arrangement for providing programmed sessions to the user. In one non-limiting embodiment, the present disclosure is not limited above mentioned spatial arrangements and any other spatial arrangement may be selected based on the environment and space constraint.

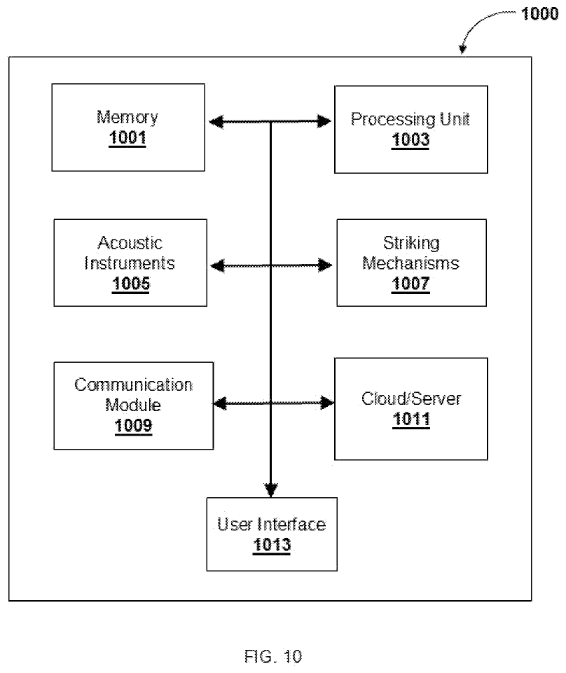

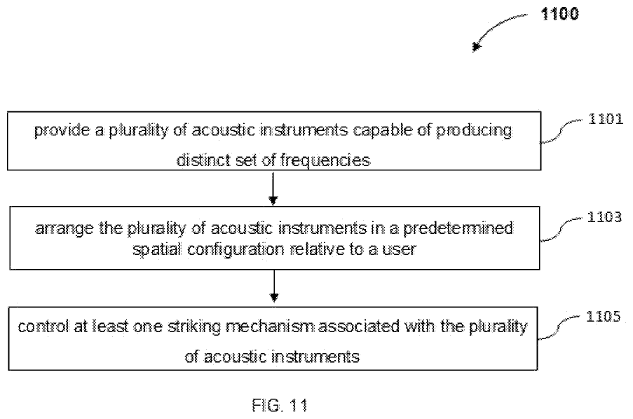

FIG. 10 illustrates a block diagram of an automated acoustic delivery apparatus 1000 for delivering automated acoustic stimulation for wellness applications, in accordance with some embodiments of the present disclosure. In an embodiment, the apparatus 1000 may be similar to the automated acoustic delivery apparatus, as discussed in above embodiments.

In an embodiment of the present disclosure, the apparatus 1000 may comprise a processing unit 1003, a memory 1001, acoustic instruments 1005, striking mechanisms 1007, a communication module 1009, and cloud/server 1011, and user interface 1013 communicatively coupled with each other. It may be noted that, in some embodiments, the apparatus 1000 may include more or fewer components than those depicted herein.

The various components of the apparatus 1000 may be implemented using hardware, software, firmware or any combinations thereof. Further, the various components of the apparatus 1000 may be operably coupled with each other. More specifically, various components of the apparatus 1000 may be capable of communicating with each other using communication channel media (such as buses, interconnects, etc.).

In one embodiment, the processing unit 1003 may be embodied as a multi-core processor, a single core processor, or a combination of one or more multi-core processors and one or more single core processors. For example, the processing unit 1003 may be embodied as one or more of various processing devices, such as a coprocessor, a microprocessor, a controller, a digital signal processor (DSP), a processing circuitry with or without an accompanying DSP, or various other processing devices including, a microcontroller unit (MCU), a hardware accelerator, a special-purpose computer chip, or the like.

The processing unit 1003 may include one or a plurality of processors. At this time, one or a plurality of processors may be a general-purpose processor, such as a central processing unit (CPU), an application processor (AP), or the like, a graphics-only processing unit such as a graphics processing unit (GPU), a visual processing unit (VPU), and/or an AI-dedicated processor such as a neural processing unit (NPU).

In one embodiment, the memory 1001 is capable of storing machine executable instructions, referred to herein as instructions. In an embodiment, the processing unit 1003 is embodied as an executor of software instructions. As such, the processing unit 1003 is capable of executing the instructions stored in the memory 1001 to perform one or more operations described herein.

The memory 1001 can be any type of storage accessible to the processing unit 1003 to perform respective functionalities. For example, the memory 1001 may include one or more volatile or non-volatile memories, or a combination thereof. For example, the memory 1001 may be embodied as semiconductor memories, such as flash memory, mask ROM, PROM (programmable ROM), EPROM (erasable PROM), RAM (random access memory), etc. and the like.

In an embodiment of the present disclosure, the acoustic instruments may have distinct set of frequencies of sound. Each acoustic instrument may have one or more actuation means comprising striking mechanism 1007 and any other mechanism for operating the respective acoustic instrument 1005.

The acoustic instruments 1005 and corresponding actuation means are arranged in different spatial configurations, discussed in above embodiments. In an embodiment, the acoustic instruments 1005 positioned on at least one side and on at least one level relative to the user support surface, or acoustic instruments positioned in a circular or elliptical arrangement in one or more levels surrounding the user support surface. However, the present disclosure is not restricted to above configurations.

In an embodiment, the acoustic instruments 1005 comprise at least one Tibetan singing bowl. Each Tibetan singing bowl has a specifically engineered geometry and specific material compositions for generation of distinct set of frequencies of sound. The Tibetan singing bowls are interchangeable to accommodate distinct set of frequencies of sound. The processing unit 1003 may be configured to activate the at least one Tibetan singing bowl to generate beat frequencies between the set of frequencies and harmonics thereof.

In an embodiment, the striking mechanism 1007 may comprise a striking means configured to contact the associated acoustic instrument, an actuation mechanism configured to drive the striking means to strike the associated acoustic instrument with a programmable force, and a return mechanism for return of the striking means from a striking position to a rest position after striking the associated acoustic instrument, as discussed in above embodiments. In an embodiment, the actuation mechanism may comprise, but not limited to, an actuator selected from solenoid actuators, stepper motors, servo motors, and pneumatic cylinders.

In an embodiment, each striking means comprises interchangeable striking heads that includes soft striking head for generation of set of frequencies and lower-order harmonics when striking the associated acoustic instrument, and hard striking head for generation of higher-order harmonics when striking the associated acoustic instrument.

In an embodiment, the processing unit 1003 may be operatively connected to each striking mechanism 1007 and may define an activation sequence specifying activation of each striking mechanism and a temporal order of activation, control strike force for activation of each striking mechanism, regulate temporal intervals between successive activations of each striking mechanism, and execute programmed sessions comprising multiple activations according to the activation sequence.

Thus, the processing unit 1003 may control strike force, timing, and spatial sequencing of the plurality of acoustic instruments to reproducibly generate predetermined acoustic frequency relationships across the programmed sessions.

In an embodiment, the user interface 1013 may enable selection of session parameters of a programmed session. The session parameters may comprise acoustic instrument selection, sequence of activation within the acoustic instruments 1005, activation and pause timing in the sequence, and session duration.

In one non-limiting aspect, the user interface may be accessible to a user for only selecting a particular type of session e.g., session 1 for relaxation, session 2 for meditation . . . etc. In one non-limiting aspect, the user interface may be used by therapist or a professional specialized in the field of acoustics for modifying the session parameters. In one non-limiting aspect, the therapist or the professional may receive access for modification of session parameters from a network administrator.

Thus, the acoustic instruments 1005 delivers automated repeatable acoustic sessions with controlled frequency content, timing, and intensity for one or more applications comprising therapy, relaxation, wellness, meditation, stress reduction, and sound healing.

In an embodiment, the apparatus 1000 may further comprise a mounting structure selected from one of: support tables and bed-mounted frames. The acoustic instruments 1005 and the corresponding striking mechanisms 1007 are integrated with the mounting structure.

In an embodiment, the mounting structure may comprise an adjustable positioning mechanism that position the acoustic instruments 1005 in a delivery configuration adjacent to a user during sessions, and retract the acoustic instruments 1005 to a storage configuration after session completion. The adjustable positioning mechanism comprises a hydraulic, pneumatic, or motorized adjustable positioning mechanism controlled by the processing unit 1003.

In an embodiment, the cloud 1011 may provide force calibration data for each acoustic instrument and striking mechanism combination, the force calibration data relating actuator control parameters to resultant sound pressure level. The cloud 1011 may also provide force control logic for adjustment of actuator control parameters based on the force calibration data to achieve target sound pressure levels.

In an embodiment, the processing unit 1003 retrieve library of session programs from the cloud 1011 for execution of the programmed sessions, and store the library of session programs in the memory 1001 for seamless operation.

In an embodiment, the apparatus 1000 may further comprise a timer mechanism for automatic termination of sessions after a programmed duration, sound pressure level monitoring unit for maintaining acoustic output below a predetermined maximum intensity threshold, and an emergency stop mechanism accessible to a user receiving sessions to immediately halt all instrument activations upon activation.

In an embodiment, the communication module 1009 may receive, from a remote computing device, commands for operating the apparatus 1000, and transmit status information of the programmed session and the apparatus 1000 to the remote computing device.

In one non-limiting embodiment, the apparatus 1000 may also comprise plurality of other acoustic instruments comprising one or more of: percussion instruments, chimes, bells, xylophones, flutes, and string instruments and their associated actuation means for production of acoustic frequencies within a predefined range through the respective acoustic instrument.

In one non-limiting embodiment, a plurality of apparatus 1000 may be implemented together and the plurality of apparatus may be configured to deliver standardized acoustic sessions across multiple apparatuses with consistent frequencies, timing, and intensity characteristics for one or more applications, a user interface communicatively coupled to the programmable controller, the user interface. The consistent frequencies corresponds repeatable acoustic range of frequencies generated by respective acoustic instruments of the plurality of apparatus.

Thus, the apparatus 1000 addresses the limitations of manual sound therapy by offering an automated and programmable solution. The apparatus 1000 delivers consistent, scalable, and customizable therapy sessions, making it suitable for a wide range of applications, including clinical, personal, and professional environments.

FIG. 11 illustrates a flowchart of a method for delivering automated acoustic stimulation for wellness applications, in accordance with some embodiments of the present disclosure.

At step 1101, the method 1100 discloses providing a plurality of acoustic instruments capable of producing distinct set of frequencies. The acoustic instruments may be one or more of instruments discussed in above embodiments. In an exemplary embodiment, the acoustic instruments may include Tibetan singing bowl, percussion instruments, chimes, bells, xylophones, flutes, and string instruments.

At step 1103, the method 1100 discloses arranging the plurality of acoustic instruments in a predetermined spatial configuration relative to a user. For example, method 1100 may comprise arranging said instruments comprises positioning at least one acoustic instrument on a left side at one or more levels, at least one acoustic instrument on a right side relative to said user at one or more levels, and at least one acoustic instrument on at least one another side relative to the user at one or more levels. Then, the method 1100 may comprise activating the left-side, the right-side acoustic instruments, and the at least one another side acoustic instruments in a pattern selected from simultaneous bilateral or multilateral activation at the one or more levels, alternating bilateral or multilateral activation at the one or more levels, and sequential bilateral or multilateral activation at the one or more levels.

In an embodiment, the arranging the plurality of acoustic instruments in the predetermined spatial configuration may comprise positioning acoustic instruments on at least one side relative to the user, wherein the activation sequence comprises spatial patterns selected from: circular activation proceeding sequentially around said user, convergence activation proceeding from peripheral to central positions, and divergence activation proceeding from central to peripheral positions for the wellness applications.

In an embodiment, the arranging the plurality of acoustic instruments in the predetermined spatial configuration may also comprise positioning at least one acoustic instrument beneath a user support surface, providing activation that delivers combined acoustic and vibrotactile stimulation through the user support surface to the user for wellness applications. In one non-limiting embodiment, the vibrotactile coupling may be controlled by strike force, timing, or placement parameters.

In an exemplary embodiment, the method 1100 may further comprise selecting a left-side instrument with a set of frequencies having range F1, selecting a right-side instrument with a set of frequencies having range F2 such that |F1-F2| falls within a target brainwave frequency range. Then, the method 1100 comprises activating left-side and right-side instruments to generate bilateral beat frequency effects in the target brainwave frequency range for the wellness applications. The target brainwave frequency range may comprise, but not limited to, delta range, theta range, alpha range, and beta range. The frequency range F1 and F2 may be selected based on a wellness application.

However, the present disclosure is not limited to above examples of spatial configuration and any other spatial configuration known to a person skilled in the art is well within the scope of present disclosure.

At step 1105, the method 1100 discloses controlling at least one striking mechanism associated with the plurality of acoustic instruments. For controlling, the method 1100 comprises defining an activation sequence specifying activation of each striking mechanism and a temporal order of activation, controlling strike force for activation of each striking mechanism, regulating temporal intervals between successive activations of each striking mechanism, and executing programmed sessions comprising multiple activations according to the activation sequence.

In one non-limiting embodiment, the controlling may comprise controlling strike force, timing, and spatial sequencing to reproducibly generate predetermined acoustic frequency relationships across the programmed sessions. Further, the executing the programmed sessions comprises sending control signals to the striking mechanism to produce acoustic output according to the defined activation sequence, strike force parameters, temporal intervals, and session duration.

In one non-limiting embodiment, the controlling may comprise controlling strike force parameters to gradually increase from initial low intensity to target intensity during an initial portion of the session and gradually decrease from target intensity to low intensity during a final portion of said session for comfortable wellness applications.

In one non-limiting embodiment, the method 1100 may comprise delivering, using the plurality of acoustic instruments, automated repeatable acoustic sessions with controlled frequency content, timing, and intensity for one or more applications comprising therapy, relaxation, wellness, meditation, stress reduction, and sound healing

In one non-limiting embodiment, the method 1100 may comprise enabling selection of session parameters of a programmed session. The session parameters comprise acoustic instrument selection, sequence of activation within the plurality of acoustic instruments, activation and pause timing in the sequence, and session duration. In an embodiment, the selection may comprise selecting specific acoustic instrument based on their set of frequencies to achieve desired frequency characteristics for the wellness applications. For example, the selected acoustic instrument comprises at least one Tibetan singing bowl, each bowl producing a distinct set of frequencies.

In one non-limiting aspect, the user interface may be accessible to a user for only selecting a particular type of session e.g., session 1 for relaxation, session 2 for meditation . . . etc. In one non-limiting aspect, the user interface may be used by therapist or a professional specialized in the field of acoustics for modifying the session parameters. In one non-limiting aspect, the therapist or the professional may receive access for modification of session parameters from a network administrator.

In one non-limiting embodiment, the method 1100 may comprise storing session parameters in a cloud-based platform and enabling access to the stored session parameters by other systems for protocol replication across diverse wellness applications. While, in another embodiment, the method 1100 may uploading session parameters associated with the programmed session to a cloud-based platform for sharing with other users for the wellness applications, and downloading protocol parameters from the cloud-based platform for storage and local execution.

The method 1100 may comprise controlling the plurality of acoustic instruments to gradually decrease activation frequency during the session, and transitioning from higher to lower brainwave frequency ranges based on the requirement or user preference.

In one non-limiting embodiment, testing of acoustic instruments may be performed. The testing process may comprise providing a candidate acoustic instrument, measuring a set of frequencies of the candidate acoustic instrument by activating the candidate acoustic instrument with a standardized strike force, recording acoustic output with a calibrated microphone, and performing frequency analysis to identify a set of frequency components.

The, the testing of acoustic instruments may comprise determining frequency stability by repeating said measuring for predetermined iterations and calculating a standard deviation of measured set of frequencies, measuring sound pressure level consistency by repeatedly activating said instrument with identical strike force and measuring sound pressure level variation, and selecting the candidate acoustic instrument for use in wellness applications if: frequency stability exhibits, but not limited to standard deviation of ±10 Hz or less, sound pressure level exhibits, but not limited, consistency within ±5 dB, and the candidate acoustic instrument exhibits zero structural defects.

Further, the selection results may be recorded in a specification sheet comprising measured fundamental frequency, frequency stability data, and sound pressure level characteristics, wherein the acoustic instruments are selected based on an objective performance criteria ensuring consistent output for wellness applications.

The testing may further comprise measuring sustain duration by determining time elapsed from activation until acoustic output decays to a threshold level, and qualifying the candidate acoustic instrument only if sustain duration exceeds a predefined duration for use in wellness applications.

Thus, the present disclosure provides a highly customizable and automated sound therapy method, requiring minimal manual intervention. By integrating the careful selection of instruments, advanced programming of the controller, and synchronized activation of the system, the disclosure delivers tailored acoustic treatments, maximizing therapeutic efficacy and user satisfaction.

A person of ordinary skill in the art will readily ascertain that the illustrated embodiments and steps in description and drawings (FIG. 1-11) are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments.

The sequence of operations of the method 1100 need not be necessarily executed in the same order as they are presented. Further, one or more operations may be grouped together and performed in the form of a single step, or one operation may have several sub-steps that may be performed in parallel or in sequential manner. Meanwhile, the above-described method 1100 performed by the apparatus 1000.

The disclosed method 1100 with reference to FIG. 11, or one or more operations of the apparatus 1000 explained with reference to FIG. 10 may be implemented using software including computer-executable instructions stored on one or more computer-readable media (e.g., non-transitory computer-readable media, such as one or more optical media discs, volatile memory components (e.g., DRAM or SRAM), or non-volatile memory or storage components (e.g., hard drives or solid-state non-volatile memory components, such as Flash memory components) and executed on a computer (e.g., any suitable computer, such as a laptop computer, net book, Web book, tablet computing device, smart phone, or other mobile computing device). Such software may be executed, for example, on a single local computer.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” may be understood to include tangible items and exclude carrier waves and transient signals, i.e., be non-transitory. Examples include Random Access Memory (RAM), Read-Only Memory (ROM), volatile memory, non-volatile memory, hard drives, CD (Compact Disc) ROMs, DVDs, flash drives, disks, and any other known physical storage media.

In one embodiment of the present disclosure, the disclosed method provides a significant technical advancement in the field of sound therapy. This advancement lies in automating and customizing the delivery of sound therapy, which traditionally relies on manual techniques performed by skilled practitioners. The disclosure leverages a programmable system to replicate and enhance the therapeutic experience, addressing the limitations of conventional methods.

In another embodiment, the disclosure aims to address the limitations of current manual sound therapy practices, particularly those involving instruments such as Tibetan singing bowls. By automating the process, the disclosure seeks to make sound therapy more accessible, consistent, and versatile. This automated approach allows users to achieve reliable therapeutic results without requiring extensive expertise or manual intervention.

In yet another embodiment, the purpose of the disclosure is to deliver the therapeutic benefits of sound therapy in an innovative manner. The programmable nature of the disclosure reduces reliance on human expertise, enabling broader accessibility and affordability of sound therapy treatments. This ensures that individuals from diverse backgrounds can access consistent and high-quality therapy sessions.

In one specific embodiment, the disclosure can also be used in research to better understand the effects of sound therapy. By standardizing sound sequences, the system enables controlled experiments that would be challenging to perform with manual methods. Objective measurements, such as physiological data from wearables or EEGs, can be combined with subjective participant feedback to provide a holistic understanding of sound therapy's impact. For example, the system ensures precise replication of specific sound patterns, allowing for controlled studies where sound therapy is the only variable. Wearables and EEGs can monitor physiological markers such as heart rate variability, stress levels, and brainwave activity, providing quantitative insights into therapy outcomes. Data collected before and after therapy sessions can highlight measurable effects on stress levels, relaxation, focus, or sleep quality. By correlating objective data with subjective experiences, researchers can better understand the efficacy of sound therapy.

Overall, the disclosure democratizes sound therapy by making it more accessible, reliable, and adaptable. Its technical design allows for standardization, scalability, and a level of therapeutic complexity that manual methods cannot achieve. This advancement not only benefits end-users but also facilitates scientific exploration of sound therapy's therapeutic mechanisms and potential applications.

The present disclosure offers multiple advantages over the prior art and the above listed are a few examples to emphasize on some of the advantageous features. The listed advantages are to be read in a non-limiting manner.

It will be understood by those within the art that, in general, terms used herein, and are generally intended as “open” terms (e.g., the term “including” may be interpreted as “including but not limited to,” the term “having” may be interpreted as “having at least,” the term “includes” may be interpreted as “includes but is not limited to,” etc.). For example, as an aid to understanding, the detail description may contain usage of the introductory phrases “at least one” and “one or more” to introduce recitations. However, the use of such phrases may not be construed to imply that the introduction of a recitation by the indefinite articles “a” or “an” limits any particular part of description containing such introduced recitation to disclosure containing only one such recitation, even when the introductory phrases “one or more” or “at least one” and indefinite articles such as “a” or “an” (e.g., “a” and/or “an” may typically be interpreted to mean “at least one” or “one or more”) are included in the recitations; the same holds true for the use of definite articles used to introduce such recitations. In addition, even if a specific part of the introduced description recitation is explicitly recited, those skilled in the art will recognize that such recitation may typically be interpreted to mean at least the recited number (e.g., the bare recitation of “two recitations,” without other modifiers, typically means at least two recitations or two or more recitations).

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following detailed description.

### EXAMPLE OF THE DISCLOSURE

### Four-Bowl Bilateral Configuration With Programmable Strike Force Control

In this exemplary embodiment, the automated acoustic delivery apparatus comprises four Tibetan singing bowls arranged in a bilateral configuration at a single horizontal level. The bowls are positioned symmetrically relative to a standard massage table (user support surface) with dimensions of 73 inches length×28 inches width.

### Bowl Specifications:

Bowl 1 (Left Front Position):
Diameter: 8 inches (203 mm)
Height: 3.5 inches (89 mm)
Wall thickness: 2.8 mm
Mass: 650 grams
Material composition: Bronze alloy
Fundamental frequency: 258 Hz
Primary harmonics: 732 Hz, 1188 Hz
Sustain duration: 24 seconds (measured to −40 dB decay)
Position: 18 inches left of table centerline, 24 inches from head of table

Bowl 2 (Right Front Position):
Diameter: 8.5 inches (216 mm)
Height: 3.75 inches (95 mm)
Wall thickness: 3.0 mm
Mass: 720 grams
Material composition: Bronze alloy
Fundamental frequency: 262 Hz
Primary harmonics: 682 Hz, 1023 Hz
Sustain duration: 26 seconds (measured to −40 dB decay)
Position: 18 inches right of table centerline, 24 inches from head of table

Bowl 3 (Left Rear Position):
Diameter: 10 inches (254 mm)
Height: 4.25 inches (108 mm)
Wall thickness: 3.5 mm
Mass: 1,050 grams
Material composition: Bronze alloy
Fundamental frequency: 193 Hz
Primary harmonics: 538 Hz, 1133 Hz
Sustain duration: 41 seconds (measured to −40 dB decay)
Position: 18 inches left of table centerline, 48 inches from head of table

Bowl 4 (Right Rear Position):
Diameter: 11 inches (279 mm)
Height: 4.5 inches (114 mm)
Wall thickness: 4.0 mm
Mass: 1,280 grams
Material composition: Bronze alloy
Fundamental frequency: 174 Hz
Primary harmonics: 522 Hz, 1096 Hz
Sustain duration: 46 seconds (measured to −40 dB decay)
Position: 18 inches right of table centerline, 48 inches from head of table

### Bilateral Beat Frequency Characteristics:

The bilateral arrangement creates specific beat frequencies between left-right bowl pairs positioned at similar distances from the user:

Front Bowl Pair (Bowl 1 and Bowl 2):
Beat frequency: 262 Hz−258 Hz=4 Hz
Brainwave range: Theta (deep relaxation, meditation)
Acoustic characteristic: Close frequency pairing produces slow, pulsing beat pattern ideal for inducing meditative states

Rear Bowl Pair (Bowl 3 and Bowl 4):
Beat frequency: 193 Hz−174 Hz=19 Hz
Brainwave range: Beta (alert, focused attention)
Acoustic characteristic: Wider frequency separation produces faster beat pattern suitable for maintaining awareness during relaxation

This configuration provides a complementary frequency architecture wherein front bowls (258 Hz, 262 Hz) deliver theta-range beat frequencies for deep relaxation, while rear bowls (193 Hz, 174 Hz) deliver beta-range beat frequencies for grounded awareness. The apparatus can selectively activate front bowls, rear bowls, or combinations thereof to achieve desired therapeutic states ranging from alert relaxation to deep meditation.

### Striking Mechanism Specifications

Each of the four bowls is equipped with an individual striking mechanism having two interchangeable mallets sub assembly mounted on an aluminum support plate. The striking mechanism comprises:

### Main Striker Bracket:

Material: Stainless steel 304
Length of vertical link: 120 mm
Bearing housing: 8 mm internal diameter precision ball bearing
Bearing housing position: 85 mm from striking end

Striking Means (Interchangeable Mallets):

Mallet 1 (soft Strike):
Head: 35 mm diameter, cylindrical felt-covered SS 304 pipe shaped head
Felt material: Compressed wool felt, density 0.35 g/cm³, Shore hardness 25A
Shaft: 8 mm diameter aluminum rod, length 100 mm

Return Mechanism:
Gravity shifting weight: 95 grams solid steel
Weight position: Attached to bottom of vertical link

Mallet 2 (Harder Strike):
Head: 30 mm diameter, cylindrical-soft cloth covered wooden head
Cloth material: Graded cloth material
Shaft: 10 mm diameter aluminum rod, length 100 mm

Return Mechanism:
Gravity shifting weight: 85 grams solid steel
Weight position: Attached to bottom of vertical link

Actuation Mechanism:
Actuator type: Stepper motor
Rotational speed range: 70 to 200 RPM (revolutions per minute)
Control signal: PWM (Pulse Width Modulation)
Supply voltage: 24V DC

### Electronics and Software Integration

In this exemplary embodiment, the automated acoustic delivery apparatus employs a distributed electronic control architecture. A central supervisory computing unit (e.g., Raspberry Pi 5 with 7-inch touch screen attached) is configured to manage session definition, scheduling, and coordination across one or more automated acoustic delivery apparatuses. The central supervisory computing unit may comprise any suitable general-purpose or embedded computing platform and is not limited to a specific processor model or hardware configuration.

Each automated acoustic delivery apparatus comprises an independent apparatus controller, implemented using a microprocessor (Raspberry Pi Pico), that is locally responsible for controlling the striking mechanisms housed within that apparatus. The central supervisory computing unit communicates with each apparatus controller by transmitting high-level session commands, including activation sequences, timing parameters, and target actuation characteristics.

The apparatus controller translates the received high-level commands into low-level actuator control signals and communicates directly with actuator driver modules associated with respective striking mechanisms. The actuator driver modules drive the actuators to produce controlled mechanical motion of the striking mechanisms according to the programmed session parameters.

During session execution, the apparatus controller manages local timing, actuator control, and return-to-rest behavior of the striking mechanisms. For bilateral or multilateral activation patterns, synchronization commands are issued by the central supervisory computing unit to multiple apparatus controllers, which execute the commands locally to achieve substantially simultaneous activation.

This distributed control arrangement enables precise, repeatable, and scalable execution of programmed acoustic sessions while decoupling high-level session orchestration from low-level actuation control.

### Example Programmed Session: Basic Bilateral Relaxation Protocol

For this session, only soft mallet is used for striking:

Session Parameters:
Total duration: 5 minutes
Acoustic pattern: Sequential then bilateral activation

Phase 1—Sequential Activation (Minutes 0:00 to 2:30):
Activation pattern: Bowl 1→Bowl 2→Bowl 3→Bowl 4, repeating
Stepper motor RPM: 130 RPM
Strike interval: 10 seconds between strikes
Total strikes in phase: 15 strikes

Phase 2—Bilateral Paired Activation (Minutes 2:30 to 5:00):
Activation pattern: Simultaneous strikes of Bowl 1 and Bowl 2 (front pair), then Bowl 3 and Bowl 4 (rear pair), alternating
Stepper motor RPM: 100 RPM
Strike interval: 15 seconds between pairs
Total strikes in phase: 20 strikes (10 front pair, 10 rear pair)

### Implementation

The processing unit performs the programmed sequence by:
1. Loading session parameters from memory,
2. Sending PWM control signals to stepper motor drivers at specified RPM,
3. Managing timing intervals between strikes,
4. Coordinating simultaneous bilateral strikes within 5 milliseconds synchronization.

### Measured Acoustic Output

Sequential activation (Phase 1, 130 RPM):
Peak SPL per strike: 74-76 dB(A)
Frequency spectrum: Individual bowl fundamentals (258 Hz, 262 Hz, 193 Hz, 174 Hz) with harmonics
Beat frequencies: 4 Hz from front bowls, 19 Hz from rear bowls when multiple bowls sustaining simultaneously

Bilateral paired activation (Phase 2, 100 RPM):
Peak SPL per pair strike: 75-77 dB(A)
Front pair beat frequency: 4 Hz (258-262 Hz difference)
Rear pair beat frequency: 19 Hz (193-174 Hz difference)

This demonstrates reproducible acoustic output with consistent stepper motor control across the four-bowl bilateral configuration.

We claim:

1. A method for delivering automated acoustic stimulation for wellness applications, comprising:
providing a plurality of acoustic instruments capable of producing distinct set of frequencies;
arranging the plurality of acoustic instruments in a predetermined spatial configuration relative to a user;
controlling at least one striking mechanism associated with the plurality of acoustic instruments, wherein controlling comprises:
defining an activation sequence specifying activation of each striking mechanism and a temporal order of activation;
controlling strike force for activation of each striking mechanism;
regulating temporal intervals between successive activations of each striking mechanism; and
executing programmed sessions comprising multiple activations according to the activation sequence.

2. The method of claim 1,
wherein controlling comprises:
controlling strike force, timing, and spatial sequencing to reproducibly generate predetermined acoustic frequency relationships across the programmed sessions; and
delivering, using the plurality of acoustic instruments, automated repeatable acoustic sessions with controlled frequency content, timing, and intensity for one or more applications comprising therapy, relaxation, wellness, meditation, stress reduction, and sound healing, and
wherein executing the programmed sessions comprises sending control signals to the striking mechanism to produce acoustic output according to the defined activation sequence, strike force parameters, temporal intervals, and session duration.

3. The method of claim 1, further comprising:
enabling selection of session parameters of a programmed session, wherein the session parameters comprise acoustic instrument selection, sequence of activation within the plurality of acoustic instruments, activation and pause timing in the sequence, and session duration.

4. The method of claim 1, wherein the plurality of acoustic instruments comprises at least one Tibetan singing bowl, each bowl producing a distinct set of frequencies, and the method further comprising selecting specific bowls based on their set of frequencies to achieve desired frequency characteristics for the wellness applications.

5. The method of claim 1, wherein the activation sequence comprises activating multiple acoustic instruments simultaneously to create complex harmonic patterns through interaction of the set of frequencies and harmonics from multiple sources for the wellness applications.

6. The method of claim 1, further comprising:
arranging said instruments comprises positioning at least one acoustic instrument on a left side at one or more levels, at least one acoustic instrument on a right side relative to said user at one or more levels, and at least one acoustic instrument on at least one another side relative to the user at one or more levels; and
activating the left-side, the right-side acoustic instruments, and the at least one another side acoustic instruments in a pattern selected from simultaneous bilateral or multilateral activation at the one or more levels, alternating bilateral or multilateral activation at the one or more levels, and sequential bilateral or multilateral activation at the one or more levels.

7. The method of claim 1, further comprising:
selecting a left-side instrument with a set of frequencies having range F1;
selecting a right-side instrument with a set of frequencies having range F2, wherein |F1-F2| falls within a target brainwave frequency range; and
activating left-side and right-side instruments to generate bilateral beat frequency effects in the target brainwave frequency range for the wellness applications, wherein the target brainwave frequency range comprises delta range, theta range, alpha range, and beta range.

8. The method of claim 1, further comprising:
storing session parameters in a cloud-based platform; and
enabling access to the stored session parameters by other systems for protocol replication across diverse wellness applications.

9. The method of claim 1, further comprising:
controlling the plurality of acoustic instruments to gradually decrease activation frequency during the session; and
transitioning from higher to lower brainwave frequency ranges,
wherein controlling comprises:
controlling strike force parameters to gradually increase from initial low intensity to target intensity during an initial portion of the session and gradually decrease from target intensity to low intensity during a final portion of said session for comfortable wellness applications.

10. The method of claim 1, wherein arranging the plurality of acoustic instruments in the predetermined spatial configuration comprises:
positioning acoustic instruments on at least one side relative to the user, wherein the activation sequence comprises spatial patterns selected from: circular activation proceeding sequentially around said user, convergence activation proceeding from peripheral to central positions, and divergence activation proceeding from central to peripheral positions for the wellness applications.

11. The method of claim 1, further comprising:
providing a candidate acoustic instrument;
measuring a set of frequencies of the candidate acoustic instrument by:
activating the candidate acoustic instrument with a standardized strike force;
recording acoustic output with a calibrated microphone; and
performing frequency analysis to identify a set of frequency components;
determining frequency stability by repeating said measuring for predetermined iterations and calculating a standard deviation of measured set of frequencies;
measuring sound pressure level consistency by repeatedly activating said instrument with identical strike force and measuring sound pressure level variation; and
selecting the candidate acoustic instrument for use in wellness applications if:
frequency stability exhibits standard deviation of less than or equal to 10 Hz or less to a frequency threshold;
sound pressure level exhibits consistency within ±5 dB to a predefined threshold value; and
the candidate acoustic instrument exhibits no visible structural defects; and
documenting selection results in a specification sheet comprising measured fundamental frequency, frequency stability data, and sound pressure level characteristics, wherein the acoustic instruments are selected based on an objective performance criteria ensuring consistent output for wellness applications.

12. The method of claim 11, further comprising:
measuring sustain duration by determining time elapsed from activation until acoustic output decays to a threshold level, and qualifying the candidate acoustic instrument only if sustain duration exceeds a predefined duration for use in wellness applications.

13. The method of claim 11, wherein arranging the plurality of acoustic instruments in the predetermined spatial configuration comprises:
positioning at least one acoustic instrument beneath a user support surface, and wherein said activation delivers combined acoustic and vibrotactile stimulation through the user support surface to the user for wellness applications.
